(19) United States
(12) Patent Application Publication
(10) Pub. No.: US 2005/0154061 A1
(43) Pub. Date: Jul. 14, 2005

(54) Acetic acid derivatives

(75) Inventors: Hilmar Bischoff, Wuppertal (DE); Elke Dittrich-Wengenroth, Wuppertal (DE); Heike Heckroth, Wuppertal (DE); Andrea Vaupel, Riehen (CH); Michael Woltering, Hilden (DE); Stefan Weigand, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Aktiengesellschaft, Leverkusen (DE)

(21) Appl. No.: 10/492,761

(22) PCT Filed: Oct. 9, 2002
(86) PCT No.: PCT/EP02/11275

(30) Foreign Application Priority Data
Oct. 18, 2001 (DE) 101 51 390.9

(51) Int. Cl. A61K 31/195; C07C 233/51

(52) U.S. Cl. 514/563; 562/450

(57) ABSTRACT

The present invention relates to novel substituted acetic acid derivatives, to processes for their preparation and to their use in medicaments, in particular as potent PPAR-delta-activating compounds for the prophylaxis and/or treatment of cardiovascular disorders, in particular dyslipidaemias and coronary heart diseases.

The present invention relates to novel substituted acetic acid derivatives, to processes for their preparation and to their use in medicaments, in particular as potent PPAR-delta-activating compounds for the prophylaxis and/or treatment of cardiovascular disorders, in particular dyslipidaemias and coronary heart diseases.

In spite of many successful therapies, coronary heart disease (CHD) remains a serious public health problem. Treatment with statins, which inhibit HMG-CoA reductase, successfully lowers the LDL cholesterol plasma concentration, resulting in a significant reduction of the mortality of patients at risk; however, convincing treatment strategies for the therapy of patients having an unfavourable HDL/LDL cholesterol ratio and/or hypertriglyceridaemia are still not available to date.

Currently, fibrates are the only therapy options for patients of these risk groups. They act as weak agonists of the peroxisome-proliferator-activated receptor (PPAR)-alpha (Nature 1990, 347, 645-50). A disadvantage of the fibrates which have hitherto been approved is that their interaction with the receptor is only weak, requiring high daily doses and causing considerable side-effects.

For the peroxisome-proliferator-activated receptor (PPAR)-delta (Mol. Endocrinol. 1992, 6, 1634-41), first pharmacological findings in animal models indicate that potent PPAR-delta-agonists may likewise lead to improvements in the HDL/LDL cholesterol ratio and in hypertriglyceridaemia.

WO 00/23407 describes PPAR modulator for treating obesity, atherosclerosis and/or diabetes.

It was an object of the present invention to provide novel compounds suitable for use as PPAR-delta modulators.

It has now been found that compounds of the general formula (I) 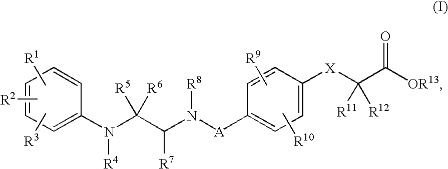
in which
A represents a bond or represents a —CH₂— or —CH₂CH₂— group,
X represents O, S or CH₂,
R¹, R² and R³ are identical or different and independently of one another represent hydrogen, (C₁-C₆)-alkyl, (C₃-C₇)-cycloalkyl, hydroxyl, (C₁-C₆)-alkoxy, amino, mono- or di-(C₁-C₆)-alkylamino, halogen, trifluoromethyl, trifluoromethoxy, nitro or cyano,
R⁴ represents hydrogen or (C₁-C₄)-alkyl,
R⁵ and R⁶ are hydrogen or together with the carbon atom to which they are attached form a carbonyl group,
R⁷ represents hydrogen or (C₁-C₄)-alkyl,
R⁸ represents straight-chain (C₅-C₁₀)-alkyl or represents a group of the formula —(CH₂)ₙ-E, in which
   E represents (C₃-C₁₂)-cycloalkyl which may be substituted up to four times by identical or different radicals from the group consisting of (C₁-C₆)-alkyl, trifluoromethyl, hydroxyl, (C₁-C₆)-alkoxy, carboxyl and (C₁-C₆)-alkoxycarbonyl, or represents 4- to 8-membered heterocyclyl which has up to two heteroatoms from the group consisting of O and S and which may be substituted up to two times by identical or different radicals from the group consisting of (C₁-C₆)-alkyl, and
   n represents the number 0, 1 or 2,
R⁹ and R¹⁰ are identical or different and independently of one another represent hydrogen, (C₁-C₆)-alkyl, (C₁-C₆)-alkoxy, trifluoromethyl or halogen,
R¹¹ and R¹² are identical or different and independently of one another represent hydrogen or (C₁-C₄)-alkyl, and
R¹³ represents hydrogen or a hydrolysable group which can be degraded to the corresponding carboxylic acid,
   and their pharmaceutically acceptable salts, hydrates and solvates,
   are pharmacologically active and can be used as medicaments or for preparing medicament formulations.

In the context of the invention, in the definition of R¹³, a hydrolysable group, means a group which, in particular in the body, causes the —C(O)OR¹³— grouping to be converted into the corresponding carboxylic acid (R¹³=hydrogen).

Such groups are, by way of example and by way of preference: benzyl, (C₁-C₆)-alkyl or (C₃-C₈)-cycloalkyl which are in each case optionally mono- or polysubstituted by identical or different substituents from the group consisting of halogen, hydroxyl, amino, (C₁-C₆)-alkoxy, carboxyl, (C₁-C₆)-alkoxycarbonyl, (C₁-C₆)-alkoxycarbonylamino or (C₁-C₆)-alkanoyloxy, or in particular (C₁-C₄)-alkyl which is optionally mono- or polysubstituted by identical or different substituents from the group consisting of halogen, hydroxyl, amino, (C₁-C₄)-alkoxy, carboxyl, (C₁-C₄)-alkoxycarbonyl, (C₁-C₄)-alkoxycarbonylamino or (C₁-C₄)-alkanoyloxy.

In the context of the invention, (C₁-C₆)-alkyl, (C₁-C₄)-alkyl and (C₁-C₃)-alkyl represent a straight-chain or branched alkyl radical having 1 to 6, 1 to 4 and 1 to 3 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl and t-butyl.

In the context of the invention, (C₅-C₁₀)-alkyl represents a straight-chain alkyl radical having 5 to 10 carbon atoms. Preference is given to a straight-chain alkyl radical having 5 to 7 carbon atoms. The following radicals may be mentioned by way of example and by way of preference: n-pentyl, n-hexyl and n-heptyl.

In the context of the invention, (C₃-C₁₂)-cycloalkyl and (C₃-C₇)-cycloalkyl represent a mono-, bi- or tricyclic cycloalkyl group having 3 to 12 carbon atoms and a mono- or bicyclic cycloalkyl group having 3 to 7 carbon atoms, respectively. The following radicals may be mentioned by way of example and by way of preference: cyclobutyl, cyclopentyl and cyclohexyl.

In the context of the invention, (C₁-C₆)-alkoxy, (C₁-C₄)-alkoxy and (C₁-C₃)-alkoxy represent a straight-chain or branched alkoxy radical having 1 to 6, 1 to 4 and 1 to 3 carbon atoms, respectively. Preference is given to a straight-chain or branched alkoxy radical having 1 to 4 carbon atoms. The following radicals may be mentioned by way of example and by way of preference: methoxy, ethoxy, n-propoxy, isopropoxy, t-butoxy, n-pentoxy and n-hexoxy.

In the context of the invention, (C₁-C₆)-alkoxycarbonyl represents a straight-chain or branched alkoxy radical having 1 to 6 carbon atoms which is attached via a carbonyl group. Preference is given to a straight-chain or branched alkoxycarbonyl radical having 1 to 4 carbon atoms. The following radicals may be mentioned by way of example and by way of preference: methoxycarbonyl, ethoxycarbonyl, n-propoxycarbonyl, isopropoxycarbonyl and t-butoxycarbonyl.

In the context of the invention, (C₁-C₆)-alkoxycarbonylamino represents an amino group having a straight-chain or branched alkoxycarbonyl substituent which has 1 to 6 carbon atoms in the alkoxy radical and is attached via the carbonyl group. Preference is given to an alkoxycarbonylamino radical having 1 to 4 carbon atoms. The following radicals may be mentioned by way of example and by way of preference: methoxycarbonylamino, ethoxycarbonylamino, n-propoxycarbonylamino and t-butoxycarbonylamino.

In the context of the invention, (C₁-C₆)-alkanoyloxy represents a straight-chain or branched alkyl radical having 1 to 6 carbon atoms which carries a doubly attached oxygen atom in the one-position and is attached in the one-position via a further oxygen atom. The following radicals may be mentioned by way of example and by way of preference: acetoxy, propionyloxy, n-butyryloxy, isobutyryloxy, pivaloyloxy, n-hexanoyloxy.

In the context of the invention, mono-(C₁-C₆)-alkylamino represents an amino group having one straight-chain or branched alkyl substituent of 1 to 6 carbon atoms. Preference is given to a straight-chain or branched monoalkylamino radical having 1 to 4 carbon atoms. The following radicals may be mentioned by way of example and by way of preference: methylamino, ethylamino, n-propylamino, isopropylamino, t-butylamino, n-pentylamino and n-hexylamino.

In the context of the invention, di-(C₁-C₆)-alkylamino and di-(C₁-C₄)-alkylamino represent an amino group having two identical or different straight-chain or branched alkyl substituents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

In the context of the invention, halogen includes fluorine, chlorine, bromine and iodine. Preference is given to chlorine or fluorine.

In the context of the invention, 4- to 8-membered heterocyclyl having up to 2 heteroatoms from the group consisting of O and S represents a saturated mono- or bicyclic heterocycle which is attached via a ring carbon atom. Preference is given to a 5- or 6-membered saturated heterocycle having one oxygen atom as heteroatom. The following radicals may be mentioned by way of example and by way of preference: tetrahydrofuran-3-yl, tetrahydropyran-3-yl and tetrahydropyran-4-yl.

Depending on the substitution pattern, the compounds according to the invention can exist in stereoisomeric forms which are either like image or mirror image (enantiomers) or which are not like image and mirror image (diastereomers). The invention relates both to the enantiomers or diastereomers and to their respective mixtures. The racemic forms, like the diastereomers, can be separated in a known manner into the stereoisomerically uniform components.

Furthermore, certain compounds can be present in tautomeric forms. This is known to the person skilled in the art, and such compounds are likewise included in the scope of the invention.

The compounds according to the invention can also be present as salts. In the context of the invention, preference is given to physiologically acceptable salts.

Pharmaceutically acceptable salts can be salts of the compounds according to the invention with inorganic or organic acids. Preference is given to salts with inorganic acids such as, for example, hydrochloric acid, hydrobromic acid, phosphoric acid or sulphuric acid or to salts with organic carboxylic or sulphonic acids such as, for example, acetic acid, propionic acid, maleic acid, fumaric acid, malic acid, citric acid, tartaric acid, lactic acid, benzoic acid, or methanesulphonic acid, ethanesulphonic acid, benzenesulphonic acid, toluenesulphonic acid or naphthalenedisulphonic acid.

Pharmaceutically acceptable salts can also be salts of the compounds according to the invention with bases, such as, for example, metal or ammonium salts. Preferred examples are alkali metal salts (for example sodium salts or potassium salts), alkaline earth metal salts (for example magnesium salts or calcium salts), and also ammonium salts which are derived from ammonia or organic amines, such as, for example, ethylamine, di- or triethylamine, ethyldiisopropylamine, monoethanolamine, di- or triethanolamine, dicyclohexylamine, dimethylaminoethanol, dibenzylamine, N-methylmorpholine, dihydroabietylamine, 1-ephenamine, methylpiperidine, arginine, lysine, ethylenediamine or 2-phenylethylamine.

The compounds according to the invention can also be present in the form of their solvates, in particular in the form of their hydrates.

Preference is given to compounds of the general formula (I)

in which

A represents a —CH₂— or —CH₂CH₂— group,
X represents O or S,
R¹ and R² are identical or different and independently of one another represent hydrogen, (C₁-C₄)-alkyl, di-(C₁-C₄)-alkylamino, chlorine, fluorine, trifluoromethyl, trifluoromethoxy, nitro or cyano,
R³ represents hydrogen,
R⁴ represents hydrogen or methyl,
R⁵ and R⁶ represent hydrogen or together with the carbon atom to which they are attached form a carbonyl group,
R⁷ represents hydrogen,
R⁸ represents (C₃-C₈)-cycloalkyl, which may be substituted up to four times by identical or different substituents from the group consisting of (C₁-C₄)-alkyl, trifluoromethyl, (C₁-C₄)-alkoxy, carboxyl and (C₁-C₄)-alkoxycarbonyl or represents 5- or 6-membered heterocyclyl which has up to two heteroatoms from the group consisting of O and S and which may be substituted up to two times by identical or different substituents from the group consisting of (C₁-C₄)-alkyl,
R⁹ represents hydrogen, (C₁-C₃)-alkyl, (C₁-C₃)-alkoxy, trifluoromethyl, fluorine or chlorine,
R¹⁰ represents hydrogen,
R¹¹ and R¹² are identical or different and independently of one another represent hydrogen or methyl, and
R¹³ represents hydrogen or represents a hydrolysable group which can be degraded to the corresponding carboxylic acid,
   and their pharmaceutically acceptable salts, hydrates and solvates.

Particular preference is given to compounds of the general formula (I)

in which

A represents a —CH₂— group,
X represents O or S,
R¹ represents hydrogen, methyl, trifluoromethyl, chlorine, fluorine, nitro or cyano,
R² represents methyl, trifluoromethyl, chlorine, fluorine, nitro or cyano,
R³ represents hydrogen,
R⁴ represents hydrogen,
R⁵ and R⁶ together with the carbon atom to which they are attached form a carbonyl group,
R⁷ represents hydrogen,
R⁸ represents cyclopentyl or cyclohexyl, each of which may be substituted by methoxy, ethoxy or up to four times by methyl, or represents 3-tetrahydrofuranyl, 3-tetrahydropyranyl or 4-tetrahydropyranyl, each of which may be mono- or disubstituted by methyl,
R⁹ represents methyl,
R¹⁰ represents hydrogen,
R¹¹ and R¹² both represent hydrogen or represent methyl, and
R¹³ represents a hydrolysable group which can be degraded to the corresponding carboxylic acid, or in particular represents hydrogen,
   and their pharmaceutically acceptable salts, hydrates and solvates.

The general or preferred radical definitions listed above apply both to the end products of the formula (I) and, correspondingly, to the starting materials and intermediates required in each case for the preparation.

The individual radical definitions given in the respective combinations or preferred combinations of radicals are, independently of the respective given combinations of radicals, also replaced by any radical definitions of other combinations.

Of particular importance are compounds of the formula (I) in which R⁴ represents hydrogen or methyl and R⁷ represents hydrogen.

Of particular importance are compounds of the formula (I) in which R⁵ and R⁶ together with the carbon atom to which they are attached form a carbonyl group.

Of particular importance are compounds of the formula (IA) 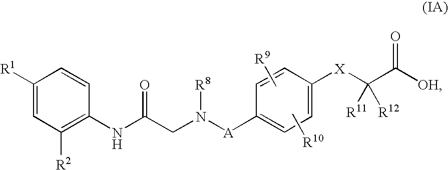
in which
R¹ and R² are identical or different and independently of one another represent methyl, trifluoromethyl, fluorine, chlorine, nitro or cyano, and
A, X, R⁸, R⁹, R¹⁰, R¹¹ and R¹² are each as defined above.

Moreover, we have found a process for preparing the compounds of the general formula (I) according to the invention, which process is characterized in that
[A] compounds of the general formula (II) 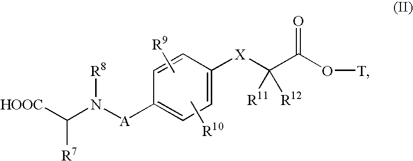
   in which
   A, X, R⁷, R⁸, R⁹, R¹⁰, R¹¹ and R¹² are as defined above and
   T represents benzyl, (C₁-C₆)-alkyl or a polymeric support suitable for solid-phase synthesis,
   are initially reacted, with activation of the carboxylic acid group in (II), with compounds of the general formula (III) 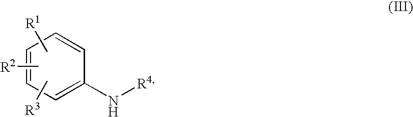
   in which
   R¹, R², R³ and R⁴ are as defined above
   to give compounds of the general formula (Ia) 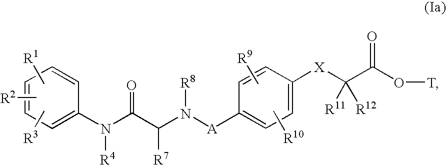
   in which
   A, X, T, R¹, R², R³, R⁴, R⁷, R⁸, R⁹, R¹⁰, R¹¹ and R¹² are as defined above, or
[B] compounds of the general formula (IV) 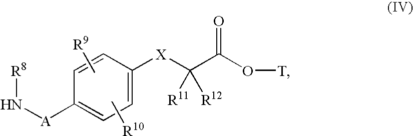
   in which
   A, X, T, R⁸, R⁹, R¹⁰, R¹¹ and R¹² are as defined above
   are reacted in the presence of a base with compounds of the general formula (V) 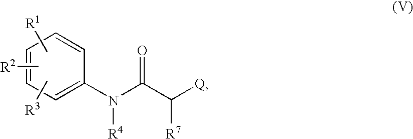
   in which
   R¹, R², R³, R⁴ and R⁷ are as defined above and
   Q represents a suitable leaving group, such as, for example, halogen, mesylate or tosylate, preferably bromine or iodine,
   likewise to give compounds of the general formula (Ia),
      the compounds of the general formula (Ia) are then, if appropriate, converted by known methods for the reduction of amides into compounds of the general formula (Ib) 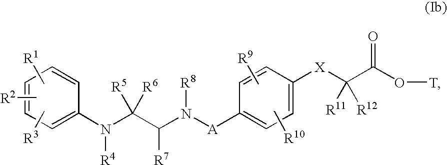
      in which
A, X, T, R¹, R², R³, R⁴, R⁵, R⁶, R⁷, R⁸, R⁹, R¹⁰, R¹¹ and R¹² are as defined above,
   which are subsequently converted with acids or bases into the corresponding carboxylic acids of the general formula (Ic) 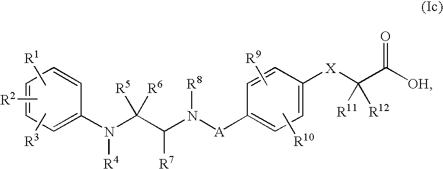
   in which
A, X, R¹, R², R³, R⁴, R⁵, R⁶, R⁷, R⁸, R⁹, R¹⁰, R¹¹ and R¹² are as defined above,
   and these are, if appropriate, further modified by known esterification methods by reaction with compounds of the general formula (VI)
   R¹³-Z (VI),
   in which
R¹³ is as defined above and
Z represents a suitable leaving group, such as, for example, halogen, mesylate or tosylate, or represents a hydroxyl group.

The process according to the invention is generally carried out at atmospheric pressure. However, it is also possible to carry out the process under elevated pressure or under reduced pressure (for example in a range of from 0.5 to 5 bar).

Solvents which are suitable for the process are customary organic solvents which do not change under the reaction conditions. These include ethers, such as diethyl ether, dioxane, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorobenzene, or ethyl acetate, pyridine, dimethyl sulphoxide, dimethylformamide, N,N′-dimethylpropyleneurea (DMPU), N-methylpyrrolidone (NMP), acetonitrile, acetone or nitromethane. It is also possible to use mixtures of the solvents mentioned.

Solvents which are preferred for process step (II)+(III)→(Ia) are dichloromethane, dimethylformamide and also dimethylformamide in combination with pyridine. For process step (IV)+(V)→(Ia), preference is given to dimethylformamide.

The process step (II)+(III)→(Ia) according to the invention is generally carried out in a temperature range of from 0° C. to +100° C., preferably from 0° C. to +40° C. The process step (IV)+(V)→(Ia) is generally carried out in a temperature range of from 0° C. to +120° C., preferably from +50° C. to +100° C.

The auxiliaries used for the amide formation in process step (II)+(III)→(Ia) are preferably customary condensing agents, such as carbodiimides, for example, N,N′-diethyl-, N,N′-dipropyl-, N,N′-diisopropyl-, N,N′-dicyclohexylcarbodiimide (DCC), N-(3-dimethylaminoisopropyl)-N′-ethylcarbodiimide hydrochloride (EDC), or carbonyl compounds, such as carbonyldiimidazole, or 1,2-oxazolium compounds, such as 2-ethyl-5-phenyl-1,2-oxazolium 3-sulphate or 2-tert-butyl-5-methyl-isoxazolium perchlorate, or acylamino compounds, such as 2-ethoxy-1-ethoxycarbonyl-1,2-dihydroquinoline, or propanephosphonic anhydride, or isobutyl chloroformate, or bis-(2-oxo-3-oxazolidinyl)-phosphoryl chloride or benzotriazolyloxy-tri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU), if appropriate in combination with further auxiliaries such as 1-hydroxybenzotriazole or N-hydroxysuccinimide, and the bases used are preferably alkali metal carbonates, for example sodium carbonate or bicarbonate or potassium carbonate or bicarbonate, or organic bases, such as trialkylamines, for example triethylamine, N-methylmorpholine, N-methylpiperidine or diisopropylethylamine, or pyridine. Particular preference is given to the combination of EDC, N-methylmorpholine and 1-hydroxybenzotriazole, of EDC, triethylamine and 1-hydroxybenzotriazole, of HATU and diisopropylethylamine and of HATU and pyridine.

Suitable bases for the reaction (IV)+(V)→(Ia) are the customary inorganic bases, such as alkali metal hydroxides, such as, for example, lithium hydroxide, sodium hydroxide or potassium hydroxide, alkali metal or alkaline earth metal carbonates, such as sodium carbonate, potassium carbonate, calcium carbonate or caesium carbonate, or sodium bicarbonate or potassium bicarbonate, or organic bases, such as trialkylamines, for example triethylamine, N-methylmorpholine, N-methylpiperidine or diisopropylethylamine. Preference is given to sodium bicarbonate.

The hydrolysis of the carboxylic acid esters in the process step (Ia) or (Ib)→(Ic) is carried out by customary methods by treating the esters in inert solvents with bases, the salts that are initially formed being converted by treatment with acid into the free carboxylic acids. In the case of the tert-butyl esters, the hydrolysis is preferably carried out using acids.

Suitable solvents for the hydrolysis of the carboxylic acid esters are water or the organic solvents which are customary for ester cleavage. These preferably include alcohols, such as methanol, ethanol, propanol, isopropanol or butanol, or ethers, such as tetrahydrofuran or dioxane, dimethylformamide, dichloromethane or dimethyl sulphoxide. It is also possible to use mixtures of the solvents mentioned. Preference is given to water/tetrahydrofuran and, in the case of the reaction with trifluoroacetic acid, to dichloromethane and, in the case of hydrogen chloride, to tetrahydrofuran, diethyl ether, dioxane or water.

Bases suitable for the hydrolysis are the customary inorganic bases. These preferably include alkali metal hydroxide or alkaline earth metal hydroxide, such as, for example, sodium hydroxide, lithium hydroxide, potassium hydroxide or barium hydroxide, or alkali metal carbonates, such as sodium carbonate or potassium carbonate, or sodium bicarbonate. Particular preference is given to using sodium hydroxide or lithium hydroxide.

Suitable acids are, in general, trifluoroacetic acid, sulphuric acid, hydrogen chloride, hydrogen bromide and acetic acid, or mixtures thereof, if appropriate with addition of water. Preference is given to hydrogen chloride or trifluoroacetic acid in the case of the tert-butyl esters and to hydrochloric acid in the case of the methyl esters.

In the case of compounds of the general formula (Ia) or (Ib) prepared by solid-phase synthesis and attached to a polymeric support via the carboxylic acid group, the cleavage from the resin to give the compounds of the general formula (Ic) is likewise carried out by the above-described customary methods for carboxylic acid ester hydrolysis. Here, preference is given to using trifluoroacetic acid.

When carrying out the hydrolysis, the base or the acid is generally employed in an amount of from 1 to 100 mol, preferably from 1.5 to 40 mol, based on 1 mole of the ester.

The hydrolysis is generally carried out in a temperature range of from 0° C. to +100° C., preferably from 0° C. to +50° C.

The compounds of the general formula (II) are novel, and they can be prepared by initially
[a]reacting compounds of the general formula (VII) 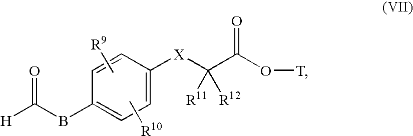
   in which
X, T, R⁹, R¹⁰, R¹¹ and R¹² are as defined above and
B represents a bond or a methylene group
   in the presence of a suitable reducing agent with compounds of the general formula (VIII)
   R¹⁴—NH₂ (VIII),
   in which
R¹⁴ [a-1] has the meaning of R⁸ given above or
   [a-2] represents a group of the formula 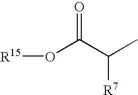
      in which
      R⁷ is as defined above and
      R¹⁵ represents (C₁-C₄)-alkyl or trimethylsilyl,
         to give compounds of the general formula (IX) 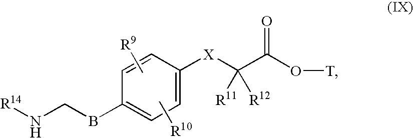
         in which
B, X, T, R⁹, R¹⁰, R¹¹, R¹² and R¹⁴ are as defined above,
   then reacting these compounds in the presence of a base with compounds of the general formula (X)
   R¹⁶—Y (X),
   in which
R¹⁶ in the case of process variant [a-1] represents a group of the formula 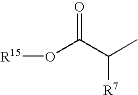
   in which R⁷ and R¹⁵ are each as defined above or,
   in the case of process variant [a-2] has the meaning of R⁸ given above and
Y represents a suitable leaving group, such as, for example halogen, mesylate or tosylate, preferably bromine or iodine,
   to give compounds of the general formula (XI) 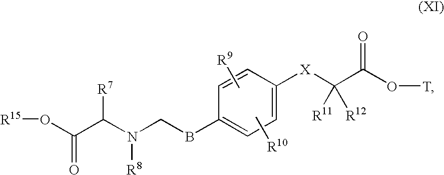
   in which
B, X, T, R⁷, R⁸, R⁹, R¹⁰, R¹¹, R¹² and R¹⁵ are as defined above,
   and finally selectively hydrolysing the carboxylic acid ester grouping —COOR¹⁵ in these compounds to the carboxylic acid, or
[b] reacting compounds of the general formula (XII) 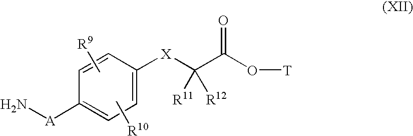
   in which
A, X, T, R⁹, R¹⁰, R¹¹ and R¹² are as defined above
   in the presence of a suitable reducing agent with compounds of the general formula (XIII)
   R¹⁷—CHO (XIII),
   in which
R¹⁷ represents straight-chain (C₄-C₉)-alkyl or represents a group of the formula —(CH₂)ₘ-E, in which
   E is as defined above and
   m represents the number 0 or 1,
      to give compounds of the general formula (XIV) 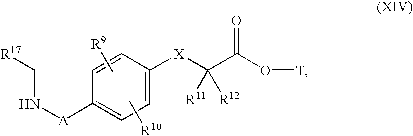
      in which
A, X, T, R⁹, R¹⁰, R¹¹, R¹² and R¹⁷ are as defined above,
   then reacting these compounds in the presence of a base with compounds of the general formula (XV) 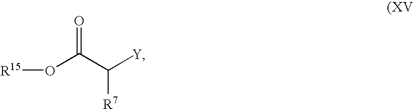
   in which
R⁷, R¹⁵ and Y are as defined above
   to give compounds of the general formula (XVI) 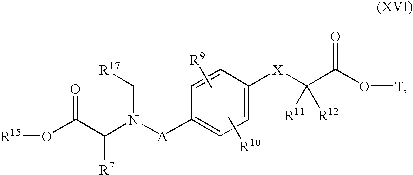
   in which
A, X, T, R⁷, R⁹, R¹⁰, R¹¹, R¹² and R¹⁷ are as defined above,
   and finally selectively hydrolysing the carboxylic acid ester grouping —COOR¹⁵ in these compounds to the carboxylic acid.

The entire process can also be carried out as solid-phase synthesis. In this case, the compounds of the general formula (VII) or (XII) are attached as carboxylic acid esters to a suitable support resin, the further reactions are carried out on solid phase and the target compound is finally cleaved off from the resin. Solid-phase synthesis and the attachment and the cleavage from the resin are customary standard techniques. To mention but one example from the extensive literature, reference is made to the publication “Linkers for Solid Phase Organic Synthesis”, Ian W. James, Tetrahedron 55, 4855-4946 (1999).

The reaction (VII)+(VIII)→(IX) or (XII)+(XIII)→(XIV) is carried out in the solvents which are customary for reductive amination and inert under the reaction conditions, if appropriate in the presence of an acid. The solvents include, for example, water, dimethylformamide, tetrahydrofuran, dichloromethane, dichloroethane, or alcohols such as methanol, ethanol, propanol, isopropanol or butanol; it is also possible to use mixtures of the solvents mentioned. Preference is given to methanol and ethanol in each case with addition of acetic acid.

Suitable reducing agents for the reaction (VII)+(VIII)→(IX) or (XII)+(XIII)→(XIV) are complex aluminium hydrides or boron hydrides, such as, for example, diisobutylaluminium hydride, sodium borohydride, sodium triacetoxyborohydride, sodium cyanoborohydride- or tetrabutylammonium borohydride, or else catalytic hydrogenation in the presence of transition metal catalysts such as, for example, palladium, platinum, rhodium or Raney nickel. Preferred reducing agents are sodium cyanoborohydride, sodium triacetoxyborohydride and tetrabutylammonium borohydride.

The reaction (VII)+(VIII)→(IX) or (XII)+(XIII)→(XIV) is generally carried out in a temperature range of from 0° C. to +40° C.

The reaction (IX)+(X)→(XI) or (XIV)+(XV)→(XVI) is carried out in the customary solvents which are inert under the reaction conditions. Preference is given to dimethylformamide, tetrahydrofuran and dioxane.

Suitable bases for the reaction (IX)+(X)→(XI) or (XIV)+(XV)→(XVI) are the customary inorganic or organic bases. Preference is given to triethylamine.

The reaction (IX)+(X)→(XI) or (XIV)+(XV)→(XVI) is generally carried out in a temperature range of from 0° C. to +100° C.

The reaction (XI)→(II) or (XVI)→(II) is carried out in the solvents which are customary for ester cleavage and inert under the reaction conditions. In the case of the ester hydrolysis, these are preferably tetrahydrofuran, dioxane and alcohols, such as methanol and ethanol, in each case in a mixture with water. In the case of the cleavage of silyl esters, preference is given to using dioxane or tetrahydrofuran.

Suitable bases for the reaction (XI)→(II) or (XVI)→(II) are, in the case of the hydrolysis, the customary inorganic bases. Preference is given to lithium hydroxide, sodium hydroxide and potassium hydroxide. In the case of the cleavage of silyl esters, preference is given to using tetrabutylammonium fluoride.

The reaction (XI)→(II) or (XVI)→(II) is generally carried out in a temperature range of from 0° C. to +100° C.

The compounds of the general formula (IV) correspond to the compounds of the general formula (IX) or (XIV) and can be prepared as described above.

The compounds of the general formulae (III), (V), (VI), (VII), (VIII), (X), (XII), (XIII) and (XV) are commercially available, known or can be prepared by customary methods [cf. P. J. Brown et al., J. Med. Chem. 42, 3785-88 (1999), for example].

The compounds of the formula (I) according to the invention have a surprising and useful spectrum of pharmacological activity and can therefore be used as versatile medicaments. In particular, they are suitable for treating coronary heart disease, for the prophylaxis of myocardial infarction and for the treatment of restenosis after coronary angioplasty or stenting. The compounds of the formula (I) according to the invention are preferably suitable for treating arteriosclerosis and hypercholesterolaemia, for increasing pathologically low HDL levels and for lowering elevated triglyceride and LDL levels. In addition, they can be used for treating obesity, diabetes, for treating metabolic syndrome (glucose intolerance, hyperinsulinaemia, dyslipidaemia and high blood pressure owing to insulin resistance), hepatic fibrosis and cancer.

The novel active compounds can be administered alone or, if required, in combination with other active compounds, preferably from the group of the CETP inhibitors, antidiabetics, antioxidants, cytostatics, calcium antagonists, antihypertensives, thyroid hormones and/or thyroid mimetics, inhibitors of HMG-CoA reductase, inhibitors of HMG-CoA reductase expression, squalene synthesis inhibitors, ACAT inhibitors, perfusion promoters, platelet aggregation inhibitors, anticoagulants, angiotensin II receptor antagonists, cholesterol absorption inhibitors, MTP inhibitors, aldolase reductase inhibitors, fibrates, niacin, anorectics, lipase inhibitors and PPAR-α and/or PPAR-γ agonists.

The activity of the compounds according to the invention can be examined, for example, in vitro by the transactivation assay described in the experimental section.

The activity of the compounds according to the invention in vivo can be examined, for example, by the tests described in the experimental section.

Suitable administration forms for administering the compounds of the general formula (I) are all customary administration forms, i.e. oral, parenteral, inhalative, nasal, sublingual, rectal, external, for example transdermal, or local, such as, for example, in the case of implants or stents. In the case of parenteral administration, particular mention has to be made of intravenous, intramuscular and subcutaneous administration, for example as a subcutaneous depot. Preference is given to oral or parenteral administration. Very particular preference is given to oral administration.

Here, the active compounds can be administered on their own or in the form of preparations. Preparations suitable for oral administration are, inter alia, tablets, capsules, pellets, sugar-coated tablets, pills, granules, solid and liquid aerosols, syrups, emulsions, suspensions and solutions. Here, the active compound has to be present in such an amount that a therapeutic effect is obtained. In general, the active compound can be present in a concentration of from 0.1 to 100% by weight, in particular from 0.5 to 90% by weight, preferably from 5 to 80% by weight. In particular, the concentration of active compound should be 0.5-90% by weight, i.e. the active compound should be present in amounts sufficient to reach the dosage range stated.

To this end, the active compounds can be converted in a manner known per se into the customary preparations. This is carried out using inert non-toxic pharmaceutically suitable excipients, auxiliaries, solvents, vehicles, emulsifiers and/or dispersants.

Auxiliaries which may be mentioned are, for example: water, non-toxic organic solvents, such as, for example, paraffins, vegetable oils (for example sesame oil), alcohols (for example ethanol, glycerol), glycols (for example polyethylene glycol), solid carriers, such as natural or synthetic ground minerals (for example talc or silicates), sugar (for example lactose), emulsifiers, dispersants (for example polyvinylpyrrolidone) and glidants (for example magnesium sulphate).

In the case of oral administration, the tablets may, of course, also contain additives such as sodium citrate, together with additives such as starch, gelatine and the like. Aqueous preparations for oral administration may furthermore comprise flavour improvers or colorants.

In the case of oral administration, preference is given to administering dosages of from 0.001 to 5 mg/kg, preferably from 0.005 to 3 mg/kg, of body weight per 24 hours.

The embodiments below illustrate the invention. The invention is not limited to the examples.

Abbreviations:

TLC Thin-layer chromatography
DCI Direct chemical ionization (in MS)
DMAP 4-N,N-Dimethylaminopyridine
ESI Electrospray ionization (in MS)
HATU O-(7-Azabenzotriazol-1-yl)-N,N, N′,N′-tetramethyluronium hexafluorophosphate
HPLC High-pressure, high-performance liquid chromatography
LC-MS Liquid-chromatography-coupled mass spectroscopy
MS Mass spectroscopy
NMR Nuclear magnetic resonance spectroscopy
Rₜ Retention time (in HPLC)
THF Tetrahydrofuran

### WORKING EXAMPLES

### Example 1

### 2-[4-[[[2-[(2,4-Dichlorophenyl)amino]-2-oxoethyl]cyclohexylamino]methyl]-2-methylphenoxy]-2-methylpropanoic Acid

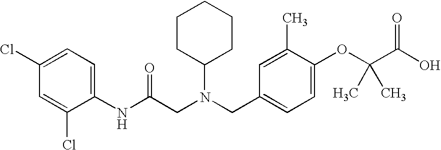 Step 1a):

### Tert-Butyl 2-(4-formyl-2-methylphenoxy)-2-methylpropanoate

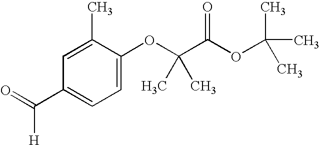

10.0 g (73.5 mmol) of 4-hydroxy-3-methylbenzaldehyde and 14.2 g (103 mmol) of potassium carbonate are initially charged in 90 ml of dimethylformamide and stirred at 90° C. for 30 min. At 50° C., 22.9 g (103 mmol) of tert-butyl 2-bromo-2-methylpropanoate are then added. After 1.5 h at 50° C., the mixture is heated to 100° C. and stirred overnight. The solvent is removed under reduced pressure. The residue is taken up in ethyl acetate and washed twice with water, twice with saturated aqueous sodium bicarbonate solution and once with saturated aqueous sodium chloride solution. The combined organic phases are dried over magnesium sulphate and concentrated. The crude product is dissolved in methanol and purified by silica gel chromatography (mobile phase: cyclohexane/ethyl acetate 10:1). This gives 5.05 g (25% of theory) of product.

LC-MS (method A): Rₜ=4.8 min

¹H-NMR (400 MHz, CDCl₃): δ=1.40 (s, 9H), 1.65 (s, 6H), 2.30 (s, 3H), 6.23 (d, 1H), 7.58 (dd, 1H), 7.67 (m, 1H), 9.83 (s, 1H).

Step 1b):

### Tert-Butyl 2-{4-[(cyclohexylamino)methyl]-2-methylphenoxy}-2-methylpropanoate

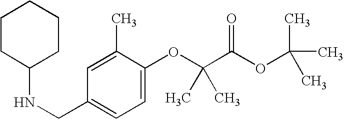

0.36 g (3.6 mmol) of cyclohexylamine and 1.0 g (3.6 mmol) of tert-butyl 2-(4-formyl-2-methylphenoxy)-2-methylpropanoate from Step 1a) are initially charged in 12 ml of dichloromethane and stirred at room temperature for 1 h. 1.2 g (5.4 mmol) of sodium triacetoxyborohydride are added, and the mixture is then stirred at room temperature overnight, and saturated aqueous sodium bicarbonate solution and dichloromethane are then added. The phases are separated and the aqueous phase is extracted with ethyl acetate. The combined organic phases are dried over magnesium sulphate and concentrated. This gives 1.29 g (99% of theory) of the crude product which is reacted without further purification.

MS (DCI): m/z=362 [M+H]⁺

¹H-NMR (200 MHz, CDCl₃): δ=1.00-1.30 (m, 4H), 1.42 (s, 9H), 1.53 (s, 6H), 1.5-2.0 (m, 6H), 2.20 (s, 3H), 2.47 (m, 1H), 3.70 (s, 2H), 6.68 (dd, 1H), 6.98 (dd, 1H), 7.07 (s, 1H).

Step 1c):

### 2-Bromo-N-(2,4-dichlorophenyl)acetamide

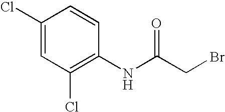

24.1 g (149 mmol) of 2,4-dichloroaniline and 20.7 ml (149 mmol) of triethylamine are initially charged in 300 ml of dichloromethane and, at 30-50° C., a solution of 30.0 g (149 mmol) of bromoacetyl bromide in 50 ml of dichloromethane is added. The mixture is stirred at room temperature for 1 h and then washed twice with water and once with saturated aqueous sodium chloride solution. The combined organic phases are concentrated and the crude product is recrystallized from ethanol. This gives 19.88 g (47% of theory) of product.

LC-MS (Method A): Rₜ=4.1 min

MS (ESI pos): m/z=282 [M+H]⁺

¹H-NMR (300 MHz, CDCl₃): δ=4.06 (s, 2H), 7.28 (dd, 1H), 7.40 (d, 1H), 8.30 (d, 1H), 8.72 (br. s, 1H).

Step 1d):

### Tert-Butyl 2-[4-[[[2-[(2,4-dichlorophenyl)amino]-2-oxoethyl]cyclohexylamino]-methyl]-2-methylphenoxy]-2-methylpropanoate

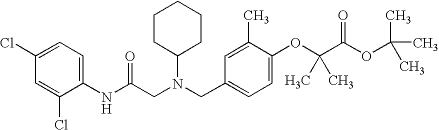

0.626 g (2.21 mmol) of 2-bromo-N-(2,4-dichlorophenyl)acetamide from Step 1c) is added to a solution of 0.800 g (2.21 mmol) of tert-butyl 2-{4-[(cyclohexylamino)methyl]-2-methylphenoxy}-2-methylpropanoate from Step 1b) and 0.205 g (2.43 mmol) of sodium bicarbonate in 12 ml of dimethylformamide, and the mixture is stirred at 90° C. for 4 h. The reaction mixture is concentrated and purified by silica gel chromatography (mobile phase: cyclohexane/ethyl acetate, gradient 95:5->70:30). This gives 0.909 g (56% of theory) of the desired product.

HPLC (Method B): Rₜ=3.69 min

¹H-NMR (300 MHz, CDCl₃): δ=1.05-1.70 (m, 6H), overlapped by 1.33 (s, 9H) and 1.47 (s, 6H), 1.77-2.0 (m, 4H), 2.14 (s, 3H), 2.57 (m, 1H), 3.23 (s, 2H), 3.61 (s, 2H), 6.61 (d, 1H), 6.98 (dd, 1H), 7.12 (br. s, 1H), 7.16 (dd, 1H), 7.33 (d, 1H), 8.40 (d, 1H), 9.93 (s, 1H).

Step 1e):

### 2-[4-[[[2-[(2,4-Dichlorophenyl)amino]-2-oxoethyl]cyclohexylamino]methyl]-2-methylphenoxy]-2-methylpropanoic Acid

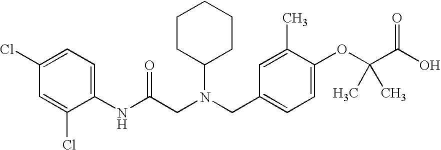

0.709 g (1.33 mmol) of tert-butyl 2-[4-[[[2-[(2,4-dichlorophenyl)amino]-2-oxoethyl]-cyclohexylamino]methyl]-2-methylphenoxy]-2-methylpropanoate from Step 1d) is dissolved in 4 ml of dichloromethane, and 4 ml of trifluoroacetic acid are added. The mixture is stirred at room temperature for 2 h and then diluted with toluene, concentrated and purified by silica gel chromatography (mobile phase: dichloromethane/methanol, gradient 98:2->95:5). This gives 0.7 g (100% of theory) of the title compound.

LC-MS (Method A): Rₜ=4.4 min

MS (ESI pos): m/z=507 [M+H]⁺

¹H-NMR (300 MHz, CDCl₃): δ=1.10-1.75 (m, 6H), overlapped by 1.60 (s, 6H), 1.87-2.18 (m, 4H), overlapped by 2.13 (s, 3H), 3.35 (m, 1H), 4.11 (s, 2H), 4.20 (s, 2H), 6.61 (d, 1H), 6.92 (dd, 1H), 7.11 (br. s, 1H), 7.16 (dd, 1H), 7.32 (d, 1H), 7.68 (d, 1H), 9.82 (s, 1H).

### Example 2

### 2-[4-[[[2-[(2,4-Dichlorophenyl)amino]-2-oxoethyl]cyclohexylamino]methyl]-2-methylphenoxy]acetic Acid

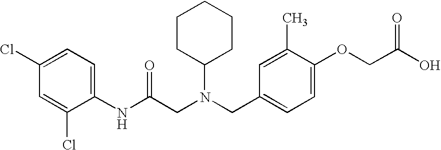 Step 2a):

### tert-Butyl(4-formyl-2-methylphenoxy)acetate

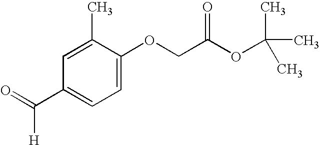

5.0 g (37 mmol) of 4-hydroxy-3-methylbenzaldehyde and 7.1 g (51 mmol) of potassium carbonate are initially charged in 45 ml of dimethylformamide and stirred at 50° C. for 30 min. At 50° C., 10.0 g (51.4 mmol) of tert-butyl 2-bromoacetate are then added. After 1 h at 50° C., the mixture is stirred at room temperature overnight. The solvent is removed under reduced pressure. The residue is taken up in ethyl acetate and washed twice with water, twice with saturated aqueous sodium bicarbonate solution and once with saturated aqueous sodium chloride solution. The combined organic phases are dried over magnesium sulphate and concentrated. The crude product (10.23 g) is reacted without further purification.

LC-MS (Method A): Rₜ=4.3 min

MS (ESI pos): m/z=195 [M+H-tBu]⁺

¹H-NMR (400 MHz, CDCl₃): δ=1.48 (s, 9H), 2.33 (s, 3H), 4.61 (s, 1H), 6.77 (d, 1H), 7.65 (d, 1H), 7.70 (br. s, 1H), 9.87 (s, 1H).

Step 2b):

### tert-Butyl{4-[(cyclohexylamino)methyl]-2-methylphenoxy}acetate

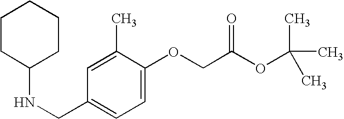

0.40 g (4.0 mmol) of cyclohexylamine and 1.0 g (4.0 mmol) of tert-butyl(4-formyl-2-methylphenoxy)acetate from Step 2a) are initially charged in 14 ml of dichloromethane and stirred at room temperature for 30 min. 1.34 g (5.99 mmol) of sodium triacetoxyborohydride are added, and the mixture is then stirred at room temperature overnight, and saturated aqueous sodium bicarbonate solution is then added. The mixture is extracted with ethyl acetate. The phases are separated and the aqueous phase is diluted with 1 M aqueous sodium hydroxide solution and re-extracted with ethyl acetate. The combined organic phases are dried over magnesium sulphate and concentrated. This gives 1.25 g (94% of theory) of crude product which is reacted without further purification.

HPLC (Method B): Rₜ=2.59 min

MS (DCI): m/z=334 [M+H]⁺

¹H-NMR (200 MHz, CDCl₃): δ=1.00-2.10 (m, 10H), overlapped by 1.48 (s, 9H), 2.28 (s, 3H), 2.49 (m, 1H), 3.70 (s, 2H), 4.50 (s, 2H), 6.61 (d, 1H), 7.05 (d, 1H), 7.10 (s, 1H).

Step 2c):

### Tert-Butyl 2-[4-[[[2-[(2,4-dichlorophenyl)amino]-2-oxoethyl]cyclohexylamino]-methyl]-2-methylphenoxy]acetate

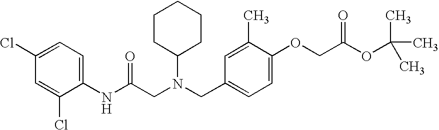

0.679 g (2.40 mmol) of 2-bromo-N-(2,4-dichlorophenyl)acetamide from Example 1/Step 1c) is added to a solution of 0.800 g (2.40 mmol) of tert-butyl {4-[(cyclohexylamino)methyl]-2-methylphenoxy}acetate from Step 2b) and 0.222 g (2.64 mmol) of sodium bicarbonate in 12 ml of dimethylformamide, and the mixture is stirred at 90° C. for 4 h. The reaction mixture is concentrated and purified by silica gel chromatography (mobile phase: cyclohexane/ethyl acetate, gradient 95:5->70:30). This gives 0.790 g (46% of theory) of the desired product.

HPLC (Method B): Rₜ=3.27 min

¹H-NMR (300 MHz, CDCl₃): δ=1.05-1.70 (m, 6H), overlapped by 1.41 (s, 9H), 1.77-2.01 (m, 4H), 2.20 (s, 3H), 2.57 (m, 1H), 3.24 (s, 2H), 3.62 (s, 2H), 4.45 (s, 1H), 6.56 (d, 1H), 7.05 (dd, 1H), 7.14 (br. s, 1H), overlapped by 7.18 (dd, 1H), 7.36 (d, 1H), 8.39 (d, 1H), 9.92 (s, 1H).

Step 2d):

### 2-[4-[[[2-[(2,4-Dichlorophenyl)amino]-2-oxoethyl]cyclohexylamino]methyl]-2-methylphenoxy]acetic Acid

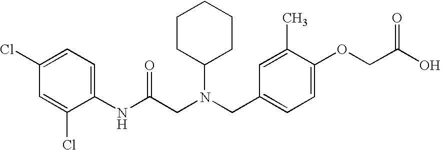

0.709 g (1.48 mmol) of tert-butyl 2-[4-[[[2-[(2,4-dichlorophenyl)amino]-2-oxoethyl]-cyclohexylamino]methyl]-2-methylphenoxy]acetate from Step 2c) is dissolved in 4 ml of dichloromethane, and 4 ml of trifluoroacetic acid are added. After 2 h of stirring at room temperature, the mixture is diluted with toluene, concentrated and purified by silica gel chromatography (mobile phase: dichloromethane/methanol, gradient 98:2->95:5). This gives 0.7 g (100% of theory) of the title compound.

LC-MS (Method A): Rₜ=3.9 min

MS (ESI pos): m/z=479 [M+H]⁺

¹H-NMR (300 MHz, CDCl₃): δ=1.10-1.74 (m, 6H), 1.86-2.18 (m, 4H), overlapped by 2.14 (s, 3H), 3.41 (m, 1H), 4.08 (s, 2H), 4.20 (s, 2H), 4.62 (s, 2H), 6.67 (d, 1H), 7.00 (d, 1H), 7.09 (s, 1H), 7.15 (dd, 1H), 7.29 (d, 1H), 7.62 (d, 1H), 9.58 (s, 1H).

### Example 3

### 2-[4-[[[2-[(2,4-Dichlorophenyl)amino]-2-oxoethyl]-(2,5-dimethylcyclopentyl)amino]methyl]-2-methylphenoxy]-2-methylpropanoic Acid

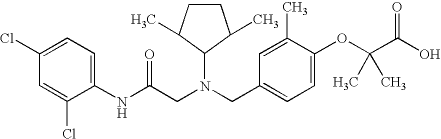 Step 3a):

### Tert-Butyl 2-(4-{[(2,5-dimethylcyclopentyl)amino]methyl}-2-methylphenoxy)-2-methylpropanoate

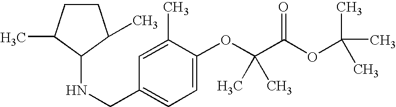

0.044 g (0.39 mmol) of 2,5-dimethylcyclopentylamine and 0.11 g (0.39 mmol) of tert-butyl 2-(4-formyl-2-methylphenoxy)-2-methylpropanoate from Example 1/Step 1a) are initially charged in 1.5 ml of dichloromethane and stirred at room temperature for 1 h. 0.13 g (0.58 mmol) of sodium triacetoxyborohydride is added, and the mixture is then stirred at room temperature for 20 h. Saturated aqueous sodium bicarbonate solution is added, and the mixture is diluted with dichloromethane. The phases are separated, the aqueous phase is extracted with ethyl acetate and the combined organic phases are dried over magnesium sulphate and concentrated. This gives 0.13 g (88% of theory) of the crude product which is reacted without further purification.

LC-MS (Method A): Rₜ=3.2 min

MS (ESI pos): m/z=376 [M+H]⁺

¹H-NMR (200 MHz, CDCl₃): δ=0.90 (d, 3H), 0.99 (d, 3H), 1.0-2.0 (m, 6H), overlapped by 1.43 (s, 9H) and 1.53 (s, 3H), 2.20 (s, 3H), 2.47 (t, 1H), 3.64 (q, 2H), 6.68 (d, 1H), 7.0 (dd, 1H), 7.10 (br. s, 1H).

Step 3b):

### Tert-Butyl 2-[4-[[[2-[(2,4-dichlorophenyl)amino]-2-oxoethyl]-(2,5-dimethylcyclopentyl)amino]methyl]-2-methylphenoxy]-2-methylpropanoate

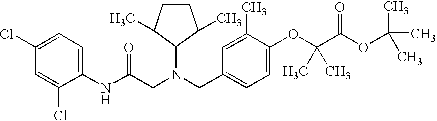

0.096 g (0.34 mmol) of 2-bromo-N-(2,4-dichlorophenyl)acetamide from Example 1/Step 1c) is added to a solution of 0.13 g (0.34 mmol) of tert-butyl 2-(4-{[(2,5-dimethylcyclopentyl)amino]methyl}-2-methylphenoxy)-2-methylpropanoate from Step 3a) and 0.032 g (0.37 mmol) of sodium bicarbonate in 3 ml of dimethylformamide, and the mixture is stirred at 90° C. for 4 h. The reaction mixture is concentrated and purified by silica gel chromatography (mobile phase: cyclohexane/ethyl acetate, gradient 99:1->70:30). Concentration gives 0.053 g (23% of theory) of the product.

TLC (cyclohexane/ethyl acetate 5:1): R_{f}=0.41

LC-MS (Method D): Rₜ=3.40 min

MS (ESI pos): m/z=577 [M+H]⁺

¹H-NMR (200 MHz, CDCl₃): δ=1.12 (t, 6H), 1.2-2.4 (m, 6H), overlapped by 1.38 (s, 9H), 1.49 (s, 3H) and 2.14 (s, 3H), 2.83 (t, 1H), 3.4-3.9 (m, 4H), 6.52 (d, 1H), 7.05 (dd, 1H), 7.19 (m, 2H), 7.37 (d, 1H), 8.32 (d, 1H), 9.72 (s, 1H).

Step 3c):

### Tert-Butyl 2-[4-[[[2-[(2,4-dichlorophenyl)amino]-2-oxoethyl]-(2,5-dimethylcyclopentyl)amino]methyl]-2-methylphenoxy]-2-methylpropanoic Acid

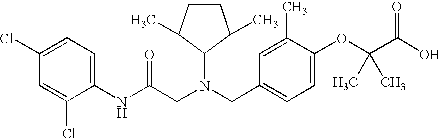

0.04 g (0.07 mmol) of tert-butyl 2-[4-[[[2-[(2,4-dichlorophenyl)amino]-2-oxoethyl]-(2,5-dimethylcyclopentyl)amino]methyl]-2-methylphenoxy]-2-methylpropanoate from Step 3b) is dissolved in 1.5 ml of dichloromethane, and 1.5 ml of trifluoroacetic acid are added. The mixture is stirred at room temperature for 2 h and then diluted with toluene, concentrated and purified by silica gel chromatography (mobile phase: dichloromethane/methanol, gradient 98:2->90:10). This gives 0.024 g (66% of theory) of the title compound.

LC-MS (Method A): Rₜ=5.52 min

MS (ESI pos): m/z=521 [M+H]⁺

¹H-NMR (300 MHz, CDCl₃): δ=1.0-2.4 (m, 6H), overlapped by 1.09 (d, 3H), 1.13 (d, 3H), 1.48 (s, 3H) and 2.12 (s, 3H), 2.75 (t, 1H), 3.3-3.85 (m, 4H), 6.70 (d, 1H), 7.0-7.3 (m, 3H), 7.44 (d, 1H), 8.38 (d, 1H), 9.65 (s, 1H).

### Example 4

### 2-{4-[(Cyclohexyl{2-[(2,4-dichlorophenyl)amino]ethyl}amino)methyl]-2-methylphenoxy}-2-methylpropanoic Acid

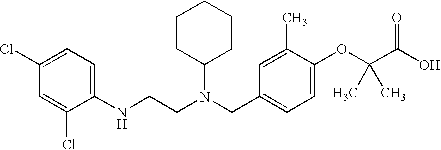 Step 4a):

### Tert-Butyl 2-{4-[(cyclohexyl{2-[(2,4-dichlorophenyl)amino]ethyl}amino)methyl]-2-methylphenoxy}-2-methylpropanoate

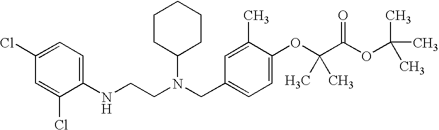

Under an atmosphere of argon, 0.16 g (0.28 mmol) of tert-butyl 2-[4-[[[2[(2,4-dichlorophenyl)amino]-2-oxoethyl]cyclohexylamino]methyl]-2-methylphenoxy]-2-methylpropanoate from Example 1/Step 1d) is initially charged in 3 ml of toluene, and 0.17 ml of borane dimethyl sulphide complex (2.0 M in tetrahydrofuran) is added. After 2 h at 110° C., the mixture is allowed to cool to room temperature, stirred with 3 ml of a 2 N aqueous sodium carbonate solution for 1 h and subsequently diluted with ethyl acetate and water. The organic phase is separated off, washed with saturated aqueous sodium chloride solution, dried over magnesium sulphate and then concentrated. The crude product is purified by silica gel chromatography (mobile phase: cyclohexane/ethyl acetate, gradient 98:2->70:30). This gives 0.085 g (54% of theory) of the title compound.

TLC (cyclohexane/ethyl acetate 5:1): R_{f}=0.59

LC-MS (Method A): Rₜ=4.0 min

MS (ESI pos): m/z=549 [M+H]⁺

¹H-NMR (300 MHz, CDCl₃): δ=1.0-1.68 (m, 6H), overlapped by 1.48 (s, 9H) and 1.55 (s, 6H), 1.7-1.92 (m, 4H), 2.13 (s, 3H), 2.48 (m, 1H), 2.80 (m, 2H), 2.92 (m, 2H), 3.51 (s, 2H), 5.18 (br. s, 1H), 6.47 (d, 1H), 6.62 (d, 1H), 6.9-7.1 (m, 3H), 7.21 (d, 1H).

Step 4b):

### 2-{4-[(Cyclohexyl{2-[(2,4-dichlorophenyl)amino]ethyl}amino)methyl]-2-methylphenoxy}-2-methylpropanoic Acid

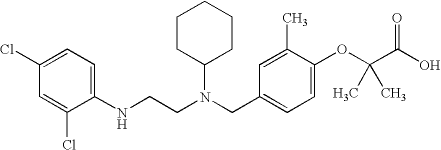

0.085 g (0.15 mmol) of tert-butyl 2-{4-[(cyclohexyl{2-[(2,4-dichlorophenyl)amino]-ethyl}amino)methyl]-2-methylphenoxy}-2-methylpropanoate from Step 4a) is dissolved in 1 ml of dichloromethane, and 1 ml of trifluoroacetic acid is added. After 2 h of stirring at room temperature, the mixture is diluted with toluene, concentrated and purified by silica gel chromatography (mobile phase: dichloromethane/methanol, gradient 98:2->95:5). This gives 0.06 g (79% of theory) of the title compound.

TLC (dichloromethane/methanol 10:1): R_{f}=0.37

LC-MS (Method A): Rₜ=3.3 min

MS (ESI neg): m/z=491 [M−H]⁺

¹H-NMR (200 MHz, CDCl₃): δ=0.8-2.2 (m, 10H), overlapped by 1.60 (s, 6H), 2.19 (s, 3H), 2.92 (m, 4H), 3.22 (br. s, 1H), 4.07 (s, 2H), 6.07 (s, H), 6.7-7.4 (m, 6H).

### Example 5

### 2-[4-[[[[2-(2,4-Dichlorophenyl)amino]-2-oxoethyl]cyclohexylamino]methyl]phenyl]-thio]-2-methylpropanoic Acid

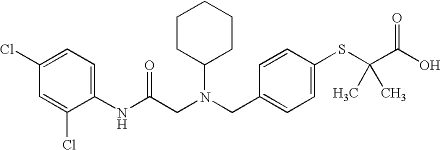 Step 5a):

### Tert-Butyl 2-[(4-bromophenyl)sulphanyl]-2-methylpropanoate

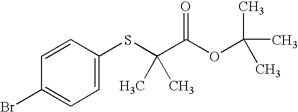

100 g (0.529 mol) of 4-bromothiophenol and 118 g (0.529 mol) of tert-butyl 2-bromisobutyrate are dissolved in 1 l of ethanol, and 29 g (0.517 mol) of potassium hydroxide are added. The mixture is stirred at reflux for 2 h and then cooled, and the potassium bromide is filtered off. The filtrate is concentrated and recrystallized from n-hexane. This gives 93.6 g (53% of theory) of the desired product as a colourless solid.

¹H-NMR (200 MHz, CDCl₃): δ=1.48 (s, 15H), 7.38 (m, 4H).

Step 5b):

### Tert-Butyl 2-[(4-formylphenyl)sulphanyl]-2-methylpropanoate

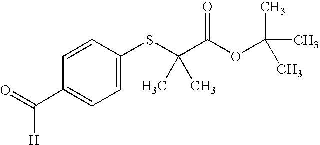

1.0 g (3.02 mmol) of tert-butyl 2-[(4-bromophenyl)sulphanyl]-2-methylpropanoate from Step 5a) are dissolved in 20 ml of THF and, at −78° C., 1.89 ml (3.02 mmol) of n-butyllithium solution in hexane are added. Directly afterwards, 0.46 ml (0.43 g, 5.92 mmol) of dimethylformamide is added and the mixture is warmed to room temperature and stirred for, another 1 h. The reaction is terminated by addition of 1 ml of 1 N hydrochloric acid and the reaction mixture is concentrated and taken up in ethyl acetate. The solution is extracted with saturated sodium bicarbonate solution and with saturated sodium chloride solution and dried over magnesium sulphate. Chromatographic purification on silica gel (mobile phase: dichloromethane) gives 0.55 g (65% of theory) of the desired product as an oil.

LC-MS: acetonitrile/30% aqueous HCl/water (gradient): Rₜ=4.86 min

MS (ESI pos): m/z=281 [M+H]⁺.

Step 5c):

### [[2-(2,4-Dichlorophenyl)amino]-2-oxoethyl]cyclohexylamine

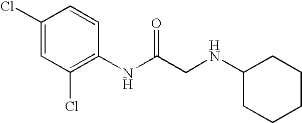

At room temperature, a solution of 1.0 g (3.534 mmol) of 2-bromo-N-(2,4-dichlorophenyl)acetamide from Example 1 (Step 1c) in 5 ml of dimethylformamide is added to a suspension of 1.752 g (17.67 mmol) of cyclohexylamine and 0.327 g (3.888 mmol) of sodium bicarbonate in 5 ml of dimethylformamide. The reaction mixture is then stirred at 90° C. for 4 h and, after cooling, poured into ice-water, and the aqueous phase is extracted three times with ethyl acetate. The combined organic extracts are washed twice with water and once with saturated sodium chloride solution and dried over sodium sulphate, and the solvent is removed under reduced pressure. This gives 0.960 g (90% of theory) of the desired product as a colourless oil.

HPLC [Kromasil C18, 0.75 ml/min, aqueous HClO₄/acetonitrile (gradient)]: Rₜ=4.15 min.

MS (DCI): m/z=301 [M+H]⁺

¹H-NMR (300 MHz, CDCl₃): δ=1.00-1.39 (m, 6H), 1.57 (s, 1H), 1.62 (m, 1H), 1.75 (m, 2H), 1.95 (m, 2H), 2.45 (m, 1H), 3.43 (s, 2H), 7.23 (dd, 1H), 7.38 (d, 1H), 8.49 (d, 1H), 10.31 (broad s, 1H).

Step 5d):

### Tert-Butyl 2-[4-[[[[2-(2,4-dichlorophenyl)amino]-2-oxoethyl]cyclohexylamino]-methyl]phenyl]thio]-2-methylpropanoate

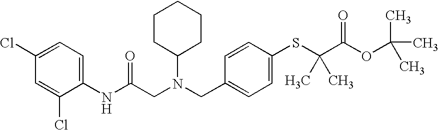

At room temperature, 87.5 mg (0.913 mmol) of sodium triacetoxyborohydride are added to a solution of 137.5 mg (0.457 mol) of [[2-(2,4-dichlorophenyl)amino]-2-oxoethyl]cyclohexylamine from Step 5c) and 160 mg (0.571 mmol) of tert-butyl 2-[(4-formylphenyl)sulphanyl]-2-methylpropanoate from Step 5b) in 5 ml of THF, and the mixture is stirred for 24 h. Saturated sodium bicarbonate solution is then added to the mixture, the phases are separated and the aqueous phase is extracted with methylene chloride. The combined organic extracts are dried over sodium sulphate, the solvent is removed under reduced pressure and the residue is purified on silica gel (mobile phase: cyclohexane/ethyl acetate 10:1). This gives 62 mg (24% of theory) of the desired product as colourless oil.

LC-MS (Method D): Rₜ=3.32 min.

MS (ESI pos): m/z=565 [M+H]⁺

¹H-NMR (400 MHz, CDCl₃): δ=1.03-1.39 (m, 5H), 1.37 (s, 9H), 1.38 (s, 6H), 1.66 (d, 1H), 1.82 (d, 2H), 1.97 (d, 2H), 2.58 (tt, 1H), 3.28 (s, 2H), 3.72 (s, 2H), 7.20 (dd, 1H), 7.32 (d, 2H), 7.35 (d, 1H), 7.42 (d, 2H), 8.42 (d, 1H), 9.86 (s, 1H).

Step 5e):

### 2-[4-[[[[2-(2,4-Dichlorophenyl)amino]-2-oxoethyl]cyclohexylamino]methyl]-phenyl]thio]-2-methylpropanoic Acid

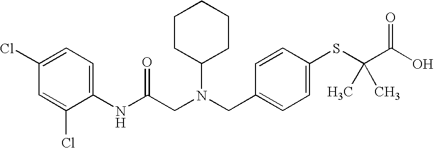

At room temperature, 277 mg (2.431 mmol) of trifluoroacetic acid are added to a solution of 55 mg (0.097 mmol) of tert-butyl-2-[4-[[[[2-(2,4-dichlorophenyl)amino]-2-oxoethyl]cyclohexylamino]methyl]phenyl]thio]-2-methylpropanoate from Step 5d) in 5 ml of dichloromethane, and the mixture is stirred for 4 h. The mixture is then concentrated under reduced pressure, the residue is taken up in ethyl acetate and the organic phase is washed twice with water and once with saturated sodium bicarbonate solution. The mixture is dried over sodium sulphate and concentrated under reduced pressure, and the residue is then purified on silica gel (mobile phase: cyclohexane/ethyl acetate 1:1). This gives 40 mg (81% of theory) of the desired product as a colourless oil.

HPLC [Kromasil C18, 0.75 ml/min, aqueous HClO₄/acetonitrile (gradient)]: Rₜ=4.51 min.

MS (ESI pos): m/z=509 [M+H]⁺

¹H-NMR (200 MHz, CDCl₃): δ=1.03-1.39 (m, 5H), 1.42 (s, 6H), 1.66 (m, 1H), 1.82 (m, 2H), 1.98 (m, 2H), 2.58 (m, 1H), 3.27 (s, 2H), 3.71 (s, 2H), 7.18 (dd, 1H), 7.28 (d, 1H), 7.32 (d, 2H), 7.42 (d, 2H), 8.37 (d, 1H), 9.82 (s, 1H).

### Example 6

### 2-[4-[[[2-[(2,4-Dimethylphenyl)amino]-2-oxoethyl]cyclohexylamino]methyl]-2-methylphenoxy]propanoic Acid

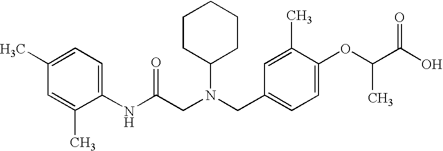

This compound was prepared by solid-phase synthesis on a polymeric support resin (Wang resin), according to reaction scheme 1 below:

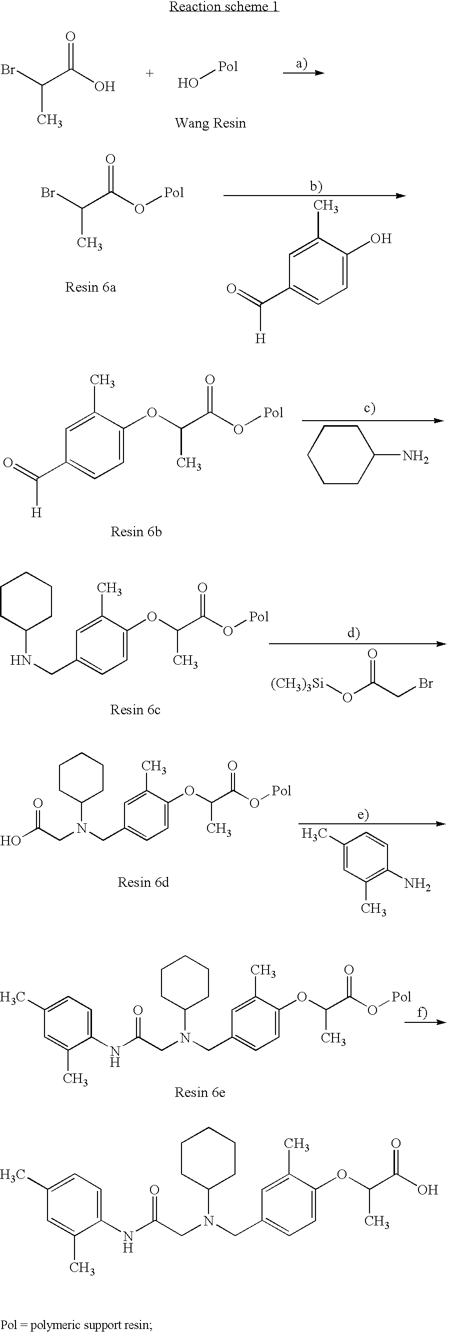

Reaction conditions: a) diisopropylcarbodiimide; DMAP, triethylamine, dichloromethane, room temperature, 20 h; b) caesium carbonate, dioxane/isopropanol 1:1, 60° C., 24 h; c) trimethyl orthoformate/dimethylformamide 1:1, room temperature, 20 h; tetrabutylammonium borohydride, acetic acid, dimethylformamide, room temperature, 20 h; d) triethylamine, dioxane, 60° C., 20 h; tetrabutylammonium fluoride, dioxane, room temperature, 1-2 h; e) HATU, pyridine/dimethylformamide 2:1, room temperature, 20 h; f) trifluoroacetic acid, dichloromethane, room temperature, 30 min.

Step 6a):

30.0 g (28.2 mmol of reactive groups; theoretical load 0.94 mmol/g) of Wang resin (from Rapp Polymere, Order No. H 1011) are suspended in 200 ml of dichloromethane. 12.9 g (84.6 mmol) of 2-bromopropanoic acid, 17.8 g (141 mmol) of diisopropylcarbodiimide and 5.17 g (42.3 mmol) of DMAP are added, and the mixture is then shaken at room temperature for 20 h. The mixture is then filtered and the resin is washed with dimethylformamide and, in an alternating manner, with methanol and dichloromethane. This gives resin 6a which is reacted without further purification.

Step 6b):

10.0 g (9.40 mmol) of resin 6a are initially charged in 100 ml of dioxane/isopropanol (1:1) and, with 21.4 g (65.8 mmol) of caesium carbonate and 8.96 g (65.8 mmol) of 4-hydroxy-3-methylbenzaldehyde, stirred at 60° C. overnight. The mixture is allowed to cool to room temperature and neutralized with 4.3 ml of acetic acid. The mixture is then filtered and the resin is washed with water, dimethylformamide and, in an alternating manner, with methanol and dichloromethane. This gives resin 6b which is reacted without further purification.

Step 6c):

2.00 g (1.88 mmol) of resin 6b and 0.932 g (9.40 mmol) of cyclohexylamine are suspended in 20 ml of trimethyl orthoformate/dimethylformamide (1:1). The mixture is shaken at room temperature for 20 h. The mixture is filtered and the resin is washed with dimethylformamide. The resin is then suspended in 20 ml of dimethylformamide, 1.93 g (7.52 mmol) of tetrabutylammonium borohydride and 1.08 ml (18.8 mmol) of acetic acid are added and the mixture is shaken at room temperature for 20 h. The mixture is filtered and the resin is washed with dimethylformamide, methanol and dichloromethane. This gives resin 6c which is directly reacted further.

Step 6d):

0.548 g (1.88 mmol) of resin 6c is suspended in 100 ml of dioxane, and 5.24 ml (37.6 mmol) of triethylamine and 4.64 ml (28.2 mmol) of trimethylsilyl bromoacetate are added. The mixture is shaken at 60° C. for 20 h. The mixture is then filtered, and the resin is washed with dimethylformamide, methanol and dichloromethane. To remove the silyl protective group, the resin is suspended in 50 ml of dioxane, and 3.8 ml (3.8 mmol) of a 1 M solution of tetrabutylammonium fluoride in THF are added. The mixture is shaken at room temperature for 1-2 h and then filtered. The resin is then washed with dimethylformamide, methanol and dichloromethane. The resulting resin 6d is directly reacted further.

Step 6e):

1.0 g (0.94 mmol) of resin 6d are suspended in 12 ml of pyridine/dimethylformamide (1:1), and 1.14 g (9.40 mmol) of 2,5-dimethylaniline and 1.07 g (2.82 mmol) of HATU are added. The mixture is shaken at room temperature for 20 h and then filtered, and the resin is washed with dimethylformamide, 30% strength acetic acid, water, dimethylformamide, methanol and dichloromethane. The resulting resin 6e is then suspended in a mixture of 12 ml of dichloromethane/trifluoroacetic acid (1:1). The mixture is shaken at room temperature for 30 min and then filtered, and the product is purified by preparative HPLC (RP-18, mobile phase: water/acetonitrile, gradient 60:40->10:90). This gives 0.14 g (33% of theory) of the title compound.

LC-MS (method D): Rₜ=1.93 min

MS (ESI pos): m/z=453 [M+H]⁺

¹H-NMR (200 MHz, DMSO-d₆): δ=1.0-2.3 (m, 10H), overlapped by 1.50 (d, 3H), 2.00 (s, 3H), 2.13 (s, 3H) and 2.22 (s, 3H), 3.48 (m, 1H), 4.34 (br. s, 2H), 4.78 (m, 3H), 6.72-7.44 (m, 6H), 9.08 (br. s, 1H).

The working examples listed below were prepared during the solid-phase synthesis of a compound library. The process exemplifies the library synthesis in MiniKans (IRORI) by the “Mix & Split” method [K. C. Nicolaou, X.-Y. Xiao, Z. Parandoosh, A. Senyei, M. P. Nova, Angew. Chem. Int. Ed. Engl. 35, 2289-2290 (1995)]. Two different methods, which are shown in reaction schemes 2 and 3, were used:

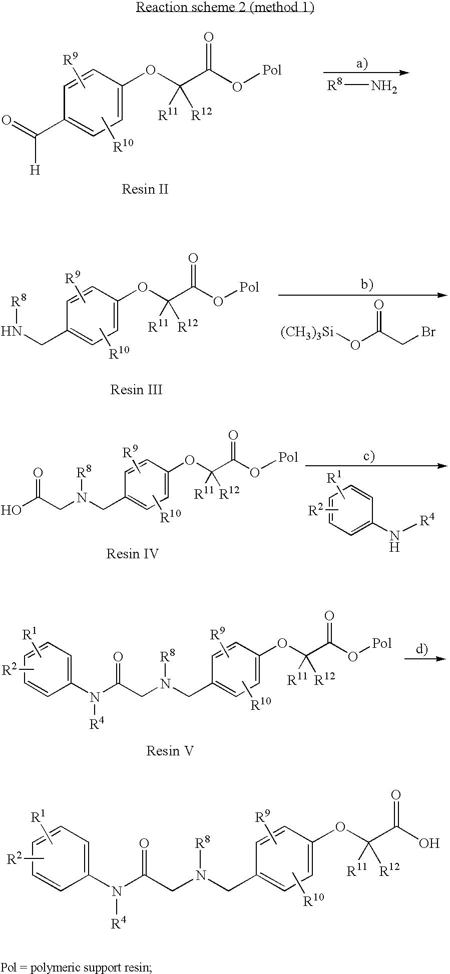

Reaction conditions: a) trimethyl orthoformate/dimethylformamide 1:1, room temperature, 12-20 h; tetrabutylammonium borohydride, acetic acid, dimethylformamide, room temperature, 20 h; b) triethylamine, dioxane, 60° C., 12-20 h; tetrabutylammonium fluoride, dioxane, room temperature, 1-2 h; c) HATU, pyridine/dimethylformamide 2:1, room temperature, 20 h; d) trifluoroacetic acid, dichloromethane, room temperature, 30 min.

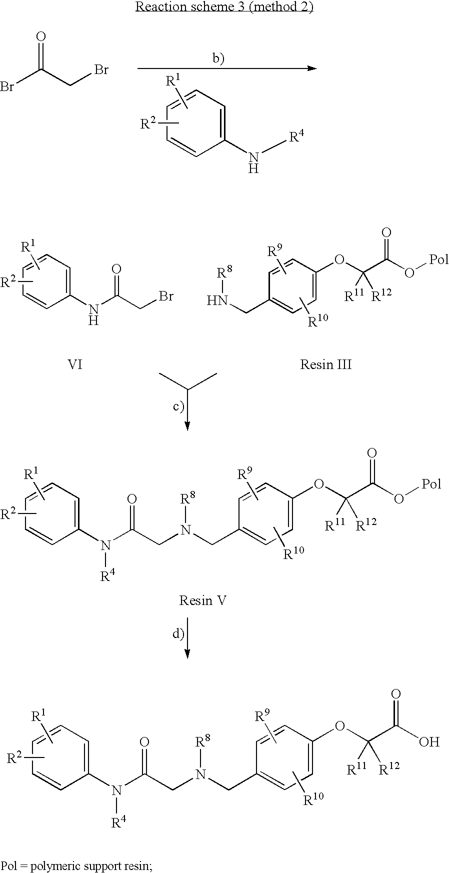

Reaction conditions: b) triethylamine, dichloromethane, 15° C.->room temperature, 1 h; c) sodium bicarbonate, dimethylformamide, 90° C., 3 h; d) trifluoroacetic acid, dichloromethane, room temperature, 30 min.

The starting resins II (reaction scheme 2) were prepared by two different methods, which are shown in reaction schemes 4 and 5:

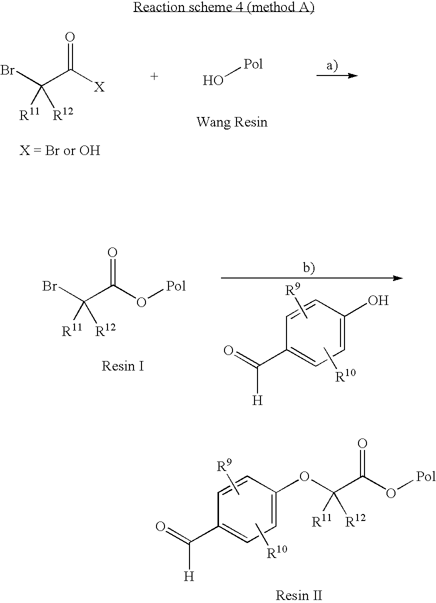

Reaction conditions: a) triethylamine, dichloromethane, −20° C.->room temperature, 24 h (X=Br) or diisopropylcarbodiimide, DMAP, triethylamine, dichloromethane, room temperature, 20 h (X=OH); b) caesium carbonate, dioxane/isopropanol 1:1, 60° C., 24 h.

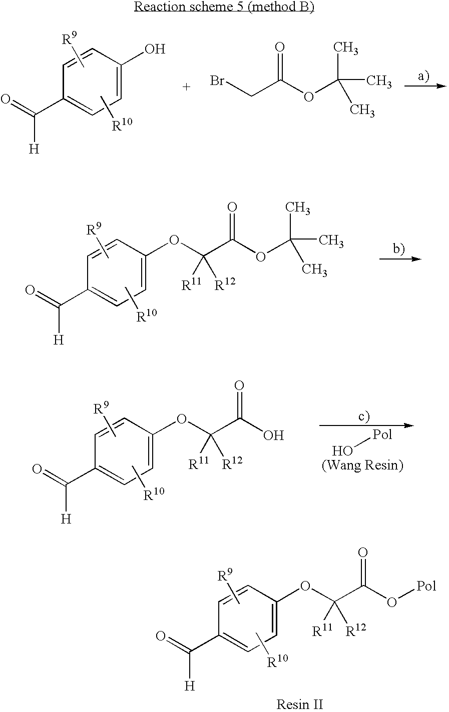

Reaction conditions: a) potassium carbonate, dimethylformamide, 50-100° C., 20 h; b) trifluoroacetic acid, dichloromethane, room temperature, 2 h; c) N,N-diisopropylcarbodiimide, DMAP, dichloromethane, room temperature, 20 h.
Preparation of the Starting Resins II by Method A: 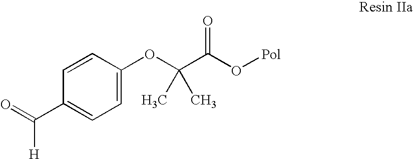
Step a):

20.0 g (18.8 mmol of reactive groups; theoretical load 0.94 mmol/g) of Wang resin (from Rapp Polymere, Order No. H 1011) are suspended in 200 ml of dichloromethane. After addition of 21.0 ml (150 mmol) of triethylamine, the mixture is cooled to −20° C., and 34.6 g (150 mmol) of 2-bromo-2-methylpropanoyl bromide are added. After 1.5 h at −20° C., the mixture is allowed to warm to room temperature and stirred for another 20 h. The mixture is then filtered and the resin is washed with dimethylformamide and, in an alternating manner, with methanol and dichloromethane. This gives resin Ia which is reacted without further purification.

Step b):

16.0 g (15.04 mmol) of resin Ia are initially charged in 160 ml of dioxane/isopropanol (1:1) and, together with 39.2 g (120 mmol) of caesium carbonate and 14.7 g (120 mmol) of 4-hydroxybenzaldehyde, stirred at 60° C. overnight. The mixture is allowed to cool to room temperature and neutralized with 6.9 ml of acetic acid. The mixture is then filtered and the resin is washed with water, dimethylformamide and, in an alternating manner, with methanol and dichloromethane. This gives resin IIa which is used without further purification for the synthesis sequence below.

The following starting resins II were prepared analogously to method A: 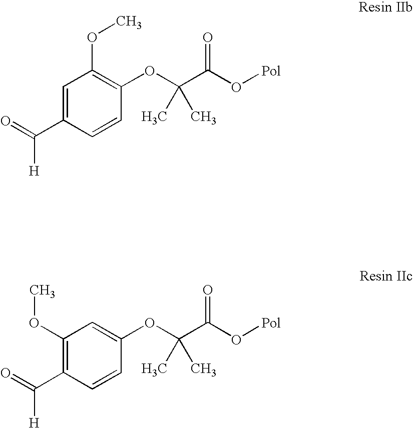
Preparation of the Starting Resins II by Method B: 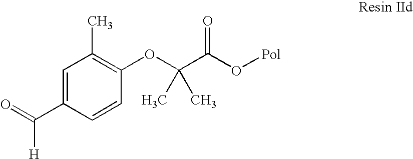
Step a):

### Tert-Butyl 2-(4-formyl-2-methylphenoxy)-2-methylpropanoate

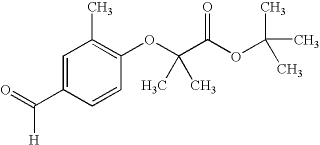

10.0 g (73.5 mmol) of 4-hydroxy-3-methylbenzaldehyde and 14.2 g (103 mmol) of potassium carbonate are initially charged in 90 ml of dimethylformamide and stirred at 50° C. for 30 min. 19.2 ml (103 mmol) of tert-butyl 2-bromoisobutyrate are added dropwise to the suspension, which is stirred at 50° C. for another 1.5 h and then heated at 100° C. for 20 h. After cooling to room temperature, the solvent is removed under reduced pressure, the residue is taken up in ethyl acetate and the mixture is extracted with water, saturated aqueous sodium bicarbonate solution and saturated aqueous sodium chloride solution. The organic phase is dried over magnesium sulphate and concentrated under reduced pressure. The crude product is purified by silica gel chromatography (mobile phase: cyclohexane/ethyl acetate 10:1). This gives 5.05 g (25% of theory) of the desired product.

LC-MS (Method A): Rₜ=4.8 min

MS (ESI pos): m/z=223 [M+H-tBu]⁺

¹H-NMR (400 MHz, CDCl₃): δ=1.40 (s, 9H), 1.68 (s, 6H), 2.29 (s, 3H), 6.74 (d, 1H), 7.58 (dd, 1H), 7.70 (br. s, 1H), 9.83 (s, 1H).

Step b):

### 2-(4-Formyl-2-methylphenoxy)-2-methylpropanoic Acid

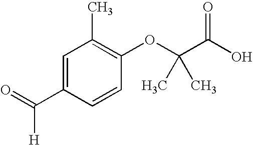

2.2 g (7.9 mmol) of tert-butyl-2-(4-formyl-2-methylphenoxy)-2-methylpropanoate are dissolved in 20 ml of dichloromethane, and 20 ml of trifluoroacetic acid are added. The mixture is stirred at room temperature for 2 h and then concentrated under reduced pressure, giving the desired compound.

LC-MS (method D): Rₜ=2.33 min

MS (ESI pos): m/z=223 [M+H]⁺
Step c):

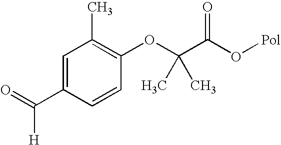

5.0 g (4.7 mmol of reactive groups; theoretical load 0.94 mmol/g) of Wang resin (from Rapp Polymere, Order No. H 1011) are suspended in 40 ml of dichloromethane. 1.98 g (8.93 mmol) of 2-(4-formyl-2-methylphenoxy)-2-methylpropanoic acid, 2.37 g (18.8 mmol) of N,N-diisopropylcarbodiimide and 0.75 g (6.1 mmol) of N,N-dimethylaminopyridine are added successively. The mixture is shaken at room temperature for 20 h and the resin is then filtered off with suction and washed repeatedly with dimethylformamide and, in an alternating manner, with methanol and dichloromethane. This gives resin, IId which is used without further purification in the synthesis sequence below.

The following starting resins II were prepared analogously to method B: 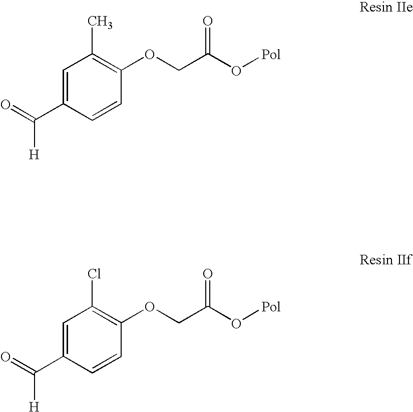
Preparation of the Bromoacetanilides VI Required for the Library Synthesis According to Reaction Scheme 3/Method 2 (See Also Example 1/Step 1c):

### VIa) 2-Bromo-N-(2,4-dimethylphenyl)acetamide

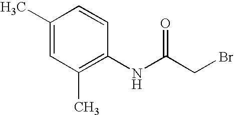

15.0 g (124 mmol) of 2,4-dimethylaniline and 17.3 ml (124 mmol) of triethylamine are initially charged in 200 ml of dichloromethane and, at 15° C., a solution of 25.0 g (124 mmol) of bromoacetyl bromide in 50 ml of dichloromethane is added. After 1 h of stirring at room temperature, the product crystallizes out. The crystals are filtered off with suction. The mother liquor is concentrated under reduced pressure. The resulting crystals are likewise filtered off with suction and dried under reduced pressure. This gives a total of 19.45 g (65% of theory) of the product.

LC-MS (Method A): Rₜ=3.54 min

MS (ESI pos): m/z=242 [M+H]⁺

### VIb) 2-Bromo-N-(2,4-difluorophenyl)acetamide

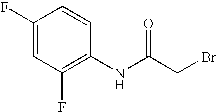

14.0 g (108 mmol) of 2,4-difluoroaniline and 15.1 ml (108 mmol) of triethylamine are initially charged in 200 ml of dichloromethane and, at 15° C., a solution of 21.9 g (108 mmol) of bromoacetyl bromide in 50 ml of dichloromethane is added. After 1 h of stirring at room temperature, water is added to the solution and the solution is washed repeatedly with water and once with saturated aqueous sodium bicarbonate solution. The combined organic phases are concentrated under reduced pressure. This causes the product to crystallize out. 22.4 g (83% of theory) of product are obtained.

LC-MS (Method A): Rₜ=3.26 min

MS (ESI pos): m/z=251 [M+H]⁺.

General Procedure for the Solid-Phase Library Synthesis According to Method 1 (Reaction Scheme 2):

a) Preparation of the Resin:

0.07-0.1 g of the resins 5b, Ia, IIb, IIc, IId, IIe and IIf (per Kan) are, as a suspension in dimethylformamide/dichloromethane (1:2), filled into IRORI Mini-Kans, washed with diethyl ether and dried.

b) Reductive Amination to Give the Resins III:

The resin compartmentalized in this manner is suspended in dichloromethane/trimethyl orthoformate (1:1) and, after addition of the amine (5-7 eq.), shaken at room temperature for 12-18 h. The resin is filtered off and washed with dimethylformamide. The resin is then suspended in dimethylformamide and glacial acetic acid (10 eq.), tetrabutylammonium borohydride (4 eq.) is added and the resin is shaken at room temperature for 6 h [alternatively to this procedure, it is possible to suspend the resin in dimethylformamide, to add tetrabutylammonium borohydride (4 eq.) and to shake at room temperature for 15 min; followed by cooling to −40° C., addition of glacial acetic acid (100 eq.) and, after warming to room temperature, shaking for 6 h]. The mixture is then filtered and the resin is washed repeatedly with methanol, dichloromethane/acetic acid (10:1), methanol, dimethylformamide, dichloromethane/diisopropylethylamine (10:1), methanol, dichloromethane and diethyl ether and finally dried under reduced pressure.

c) Alkylation with Trimethylsilyl Bromoacetate:

Under an atmosphere of argon, the separated reaction vessels are suspended in 2.5 ml of dioxane per Kan, and triethylamine (14 eq.) and trimethylsilyl bromoacetate (14 eq.) are added. The mixture is shaken at 60° C. overnight. The mixture is then filtered and the resin is washed with water, methanol, dimethylformamide, methanol, dichloromethane, methanol, dichloromethane and diethyl ether. After drying under reduced pressure, the entire reaction is repeated. The resin is finally washed with water and, twice, with dioxane.

d) Cleavage of the Trimethylsilyl Ester to Give the Resins IV.

The resin is suspended in 2.5 ml of dioxane/Kan, and tetrabutylammonium fluoride (2 eq. of a 1 M solution in THF) is added. The mixture is shaken at room temperature for 2 h and then filtered. The resin is then washed with dimethylformamide, methanol, dichloromethane and diethyl ether.

e) Amide Formation to Give the Resins V.

The resin is suspended in pyridine/dimethylformamide (2:1), and the aniline derivative (5-10 eq.) and HATU (3 eq.) are added. The mixture is shaken at room temperature for 20 h and then filtered. In some cases, this procedure has to be repeated to achieve complete conversion. The resin is then washed with 30% strength acetic acid, water, dimethylformamide, methanol, dichloromethane, methanol and dichloromethane.

f) Cleavage from the Support Resin:

The bottom of the reactors is cut open, and the reactors are, in Flex-Chem blocks, treated four times with in each case 500 μl of dichloromethane/trifluoroacetic acid (1:1). Concentration under reduced pressure gives the product in question.

### Example 7

### 2-[4-[[[2-[N-Methyl-2,4-(dimethylphenyl)amino]-2-oxoethyl]cyclohexylamino]-methyl]phenoxy]-2-methylpropanoic Acid

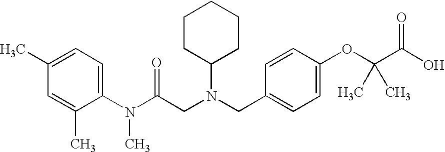

This compound was prepared using the general procedures for the library synthesis according to method 1.

LC-MS (Method C): Rₜ=3.1 min

MS (ESI pos): m/z=467 [M+H]⁺.

General Procedure for the Solid-Phase Library Synthesis According to Method 2 (Reaction Scheme 3):

a) Alkylation with Bromoacetanilides Giving the Resins V.

The separated reaction vessels with the resins III obtained according to method 1 (reaction scheme 2) are initially charged in dimethylformamide, and sodium bicarbonate (3 eq.) and the bromoacetanilide from Example 1/Step 1c), VIa or VIb (3 eq.) are added. The mixture is stirred at 90° C. for 3 h. The resin is then washed with methanol, dimethylformamide, dichloromethane, methanol, dichloromethane and diethyl ether.

b) Cleavage from the Support Resin:

The bottom of the reactors is cut open, and the reactors are treated in Flex-Chem blocks four times with in each case 500 μl of dichloromethane/trifluoroacetic acid (1:1). Concentration under reduced pressure gives the product in question.

The working examples 8-56 listed in the table below were prepared according to the general procedures for the library synthesis according to method 1 or according to method 2:

| | | | | LC-MS: | |
|---|---|---|---|---|---|
| Ex. | Synthesis | | MW | Rₜ[min] | MW found |
| No. | method | Structure | calculated | method | [M+H]⁺ |
| 8 | 1 | 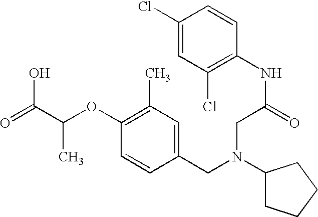 | 479.40 | 3.6 E | 479 |
| 9 | 1 | 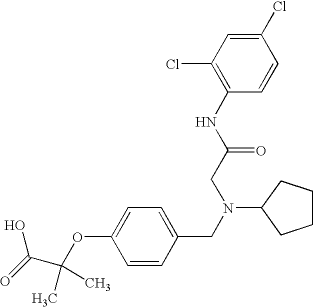 | 479.40 | 3.58 E | 479 |
| 10 | 1 | 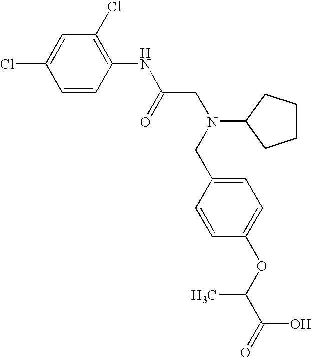 | 465.37 | 3.39 E | 465 |
| 11 | 1 | 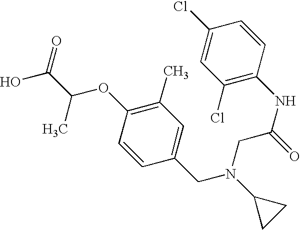 | 451.35 | 4.54 E | 451 |
| 12 | 1 | 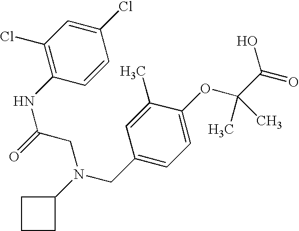 | 479.40 | 3.8 E | 479 |
| 13 | 1 | 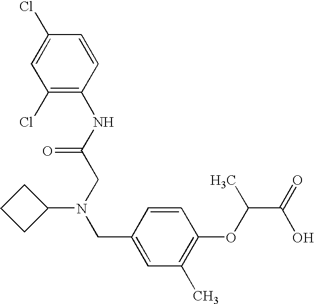 | 465.37 | 3.6 E | 465 |
| 14 | 1 | 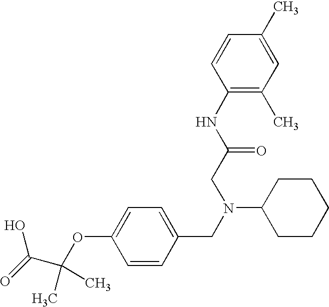 | 452.59 | 3.16 E | 453 |
| 15 | 2 | 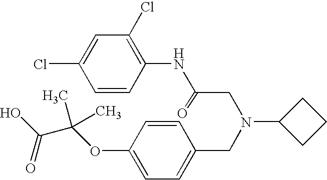 | 465.37 | 3.39 F | 465 |
| 16 | 1 | 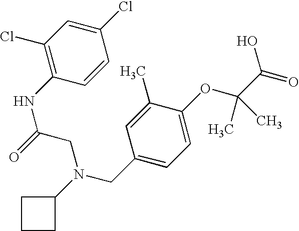 | 479.40 | 3.55 F | 479 |
| 17 | 1 | 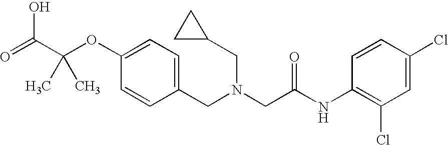 | 465.37 | 3.25 F | 465 |
| 18 | 1 | 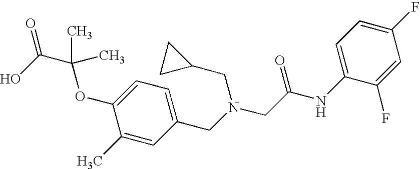 | 446.49 | 2.99 F | 447 |
| 19 | 1 | 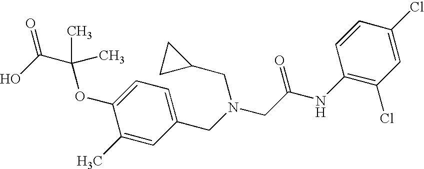 | 479.40 | 3.39 F | 479 |
| 20 | 1 | 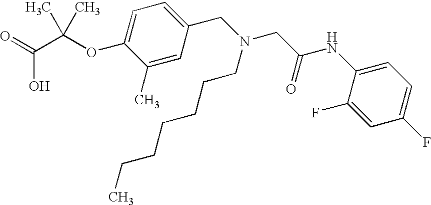 | 490.59 | 3.54 F | 491 |
| 21 | 1 | 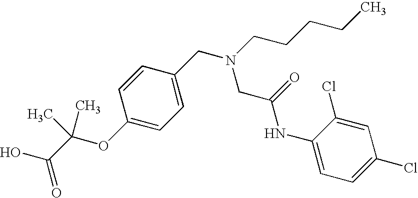 | 481.42 | 3.64 F | 481 |
| 22 | 1 | 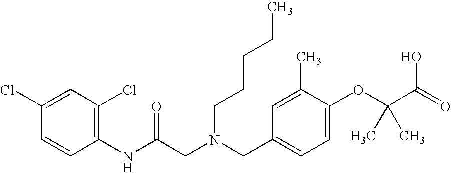 | 495.44 | 3.77 F | 495 |
| 23 | 1 | 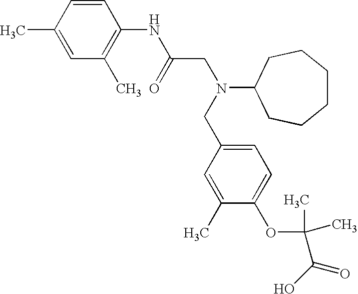 | 480.64 | 3.16 F | 481 |
| 24 | 1 | 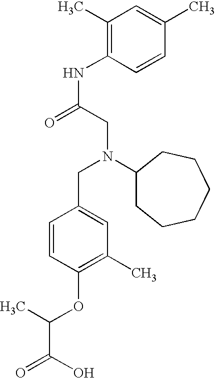 | 466.62 | 3.25 A | 466 [M]⁺ |
| 25 | 1 | 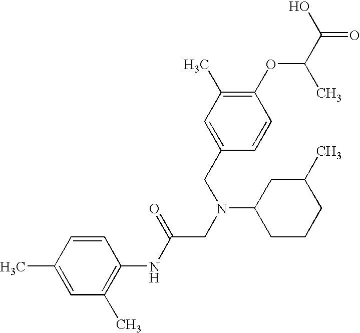 | 466.62 | 3.35 A | 467 |
| 26 | 1 | 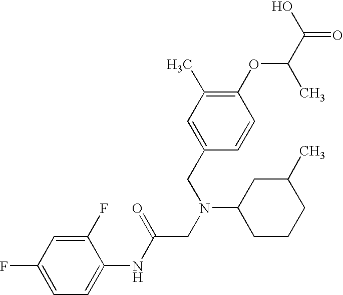 | 474.54 | 3.44 A | 474 [M]⁺ |
| 27 | 1 | 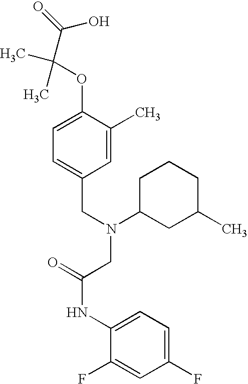 | 488.57 | 3.26 F | 489 |
| 28 | 1 | 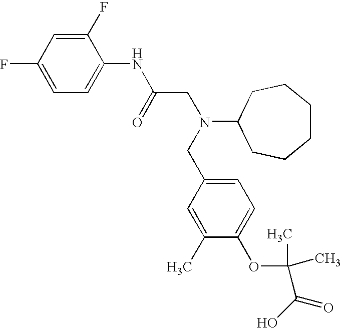 | 488.57 | 3.18 F | 489 |
| 29 | 1 | 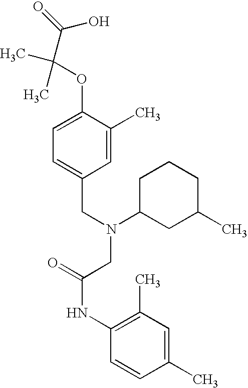 | 480.64 | 3.22 F | 481 |
| 30 | 1 | 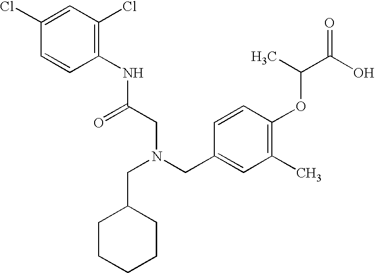 | 507.45 | 5.02 A | |
| 31 | 1 | 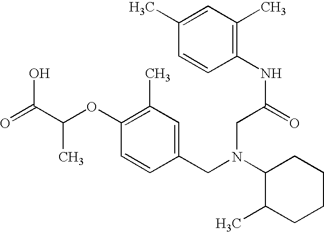 | 466.62 | 3.32 F | 468 |
| 32 | 1 | 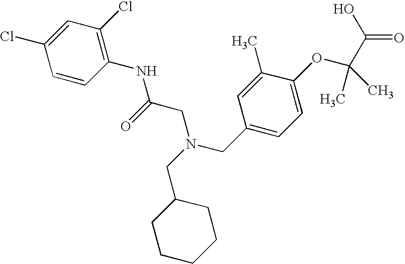 | 521.48 | 4.48 F | 521 |
| 33 | 1 | 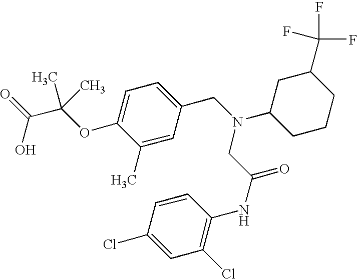 | 575.45 | 4.57 F | 575 |
| 34 | 1 | 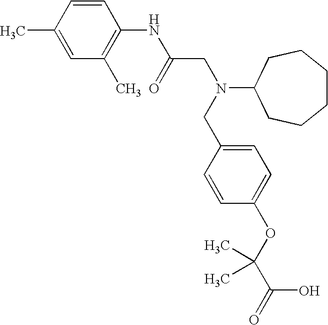 | 466.62 | 3.25 A | 467 |
| 35 | 1 | 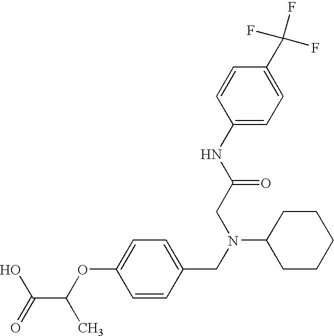 | 478.51 | 3.17 A | 478 [M]⁺ |
| 36 | 1 | 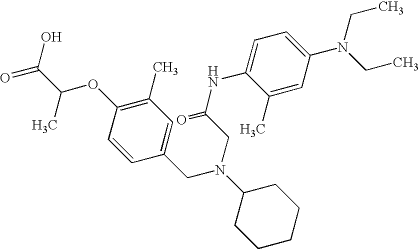 | 509.69 | 2.35 A | 510 |
| 37 | 1 | 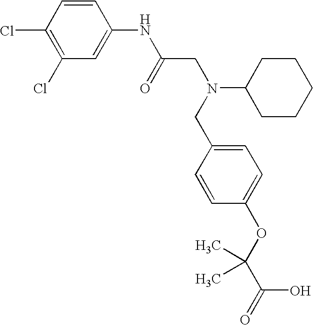 | 493.43 | 3.35 C | 493 |
| 38 | 1 | 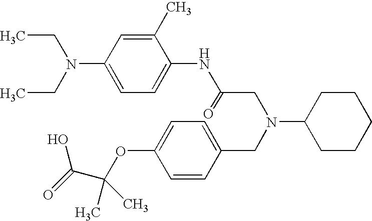 | 509.69 | 2.34 C | 510 |
| 39 | 1 | 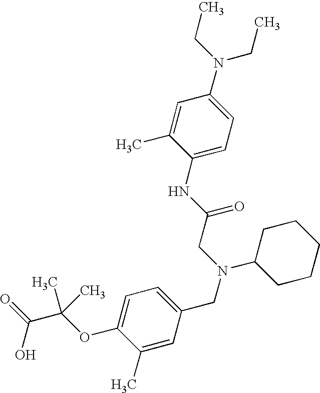 | 523.71 | 2.53 C | 524 |
| 40 | 1 | 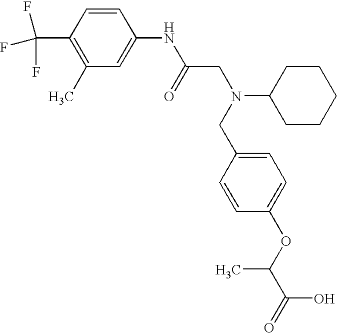 | 492.53 | 3.27 A | 492 [M]⁺ |
| 41 | 1 | 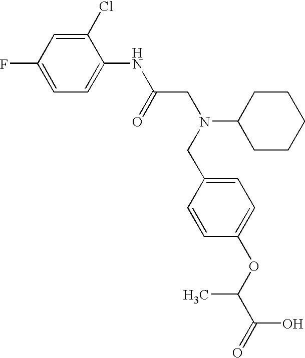 | 462.95 | 3.40 A | 463 |
| 42 | 2 | 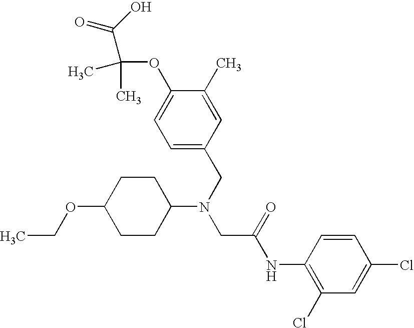 | 551.51 | 4.41 C | 551 |
| 43 | 2 | 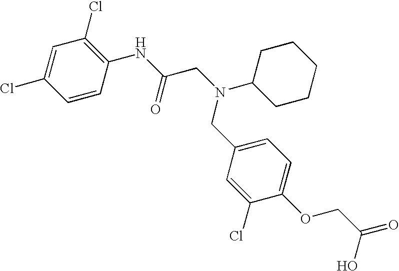 | 499.82 | 4.58 C | 499 |
| 44 | 2 | 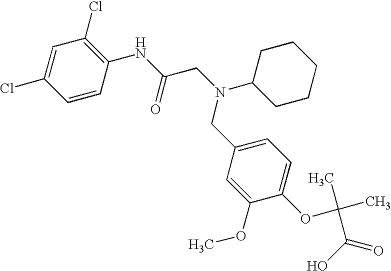 | 523.45 | 4.14 C | 523 |
| 45 | 2 | 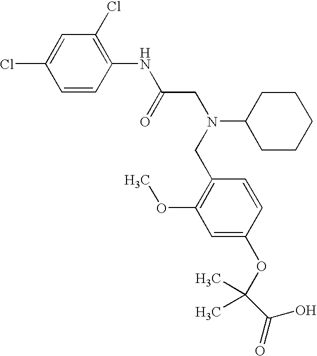 | 523.45 | 3.64 C | 523 |
| 46 | 2 | 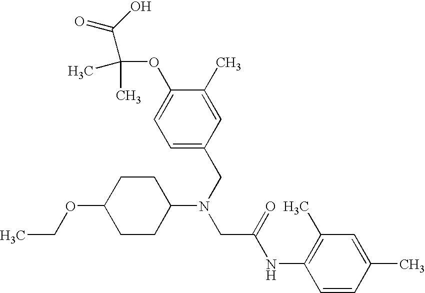 | 510.67 | 3.33 C | 511 |
| 47 | 2 | 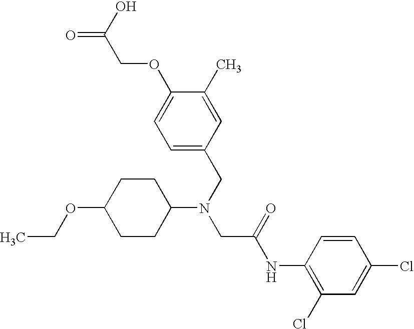 | 523.45 | 3.93 C | |
| 48 | 2 | 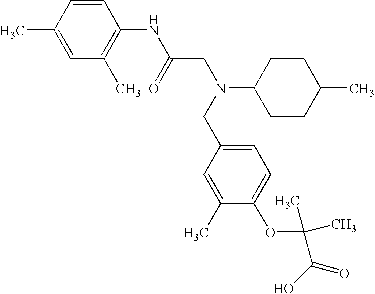 | 480.64 | 3.53 C | 481 |
| 49 | 2 | 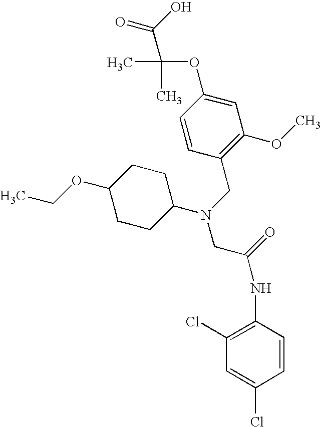 | 567.51 | 3.61 C | 567 |
| 50 | 2 | 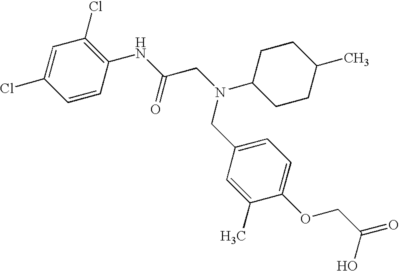 | 493.43 | 4.27 C | 493 |
| 51 | 2 | 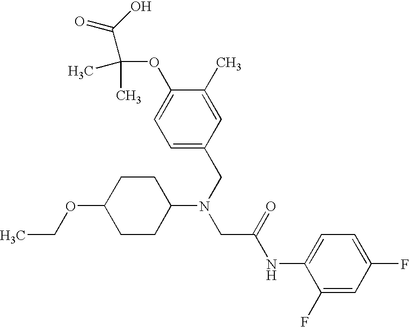 | 518.60 | 3.41 C | 519 |
| 52 | 2 | 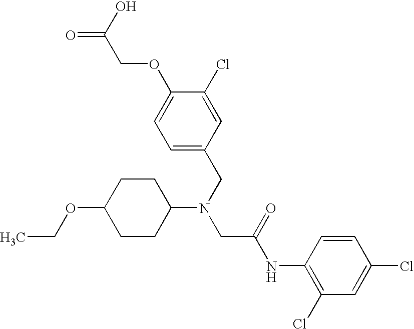 | 543.87 | 4.45 C | 545 |
| 53 | 2 | 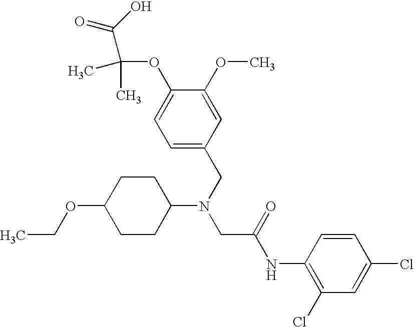 | 567.51 | 4.1 C | 567 |
| 54 | 2 | 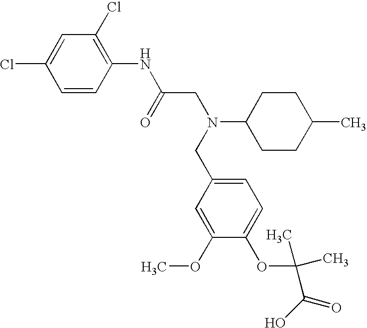 | 537.48 | 4.42 C | 537 |
| 55 | 2 | 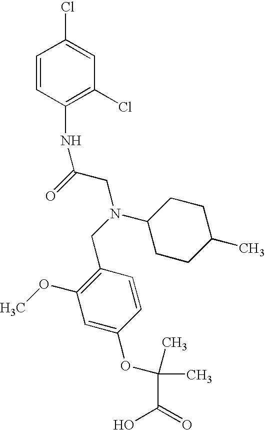 | 537.48 | 3.78 C | 537 |
| 56 | 2 | 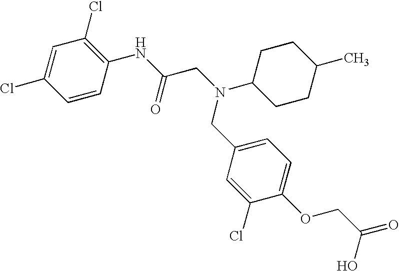 | 513.85 | 4.85 C | 513 |

LC-MS Methods:
A: Symmetry C-18, 3.5 μm, 2.1×50 mm; 70° C.; 0.5 ml/min; mobile phase A=acetonitrile+0.1% formic acid, mobile phase B=water+0.1% formic acid, gradient: 0.0 min 10% A→4 min 90% A→6 min 90% A.
B: LiChrospher 100 RP 18, 5 μm, 40° C.; 2.5 ml/min; mobile phase A=acetonitrile+0.05% trifluoroacetic acid, mobile phase B=water+0.05% trifluoroacetic acid, gradient: 0.0 min 10% A→3.0 min 90% A→4 mm 90% A.
C: Symmetry C-18, 3.5 μm, 2.1×50 mm; 40° C.; 0.5 ml/min; mobile phase A=acetonitrile+0.1% formic acid, mobile phase B=water+0.1% formic acid, gradient: 0.0 min 10% A→4 min 90% A→6 min 90% A.
D: Symmetry C-18, 5 μm, 2.1×150 mm; 70° C.; 1.2 ml/min; mobile phase A=acetonitrile, mobile phase B=water+0.3 g 30% HCl/1, gradient: 0.0 min 2% A→2.5 min 95% A→5 min 95% A.
E: Symmetry C-18, 3.5 μm, 2.1×50 mm; 24° C.;-0.75 ml/min; mobile phase A=acetonitrile+0.1% formic acid, mobile phase B=water+0.1% formic acid, gradient: 0.0 min 10% A→4 min 90% A→5.5 min 90% A.
F: YMC-ODS-AQ, 3 μm, 2.1×50 mm; room temperature; 0.8 ml/min; mobile phase A=water+0.05% formic acid, mobile phase B=acetonitrile+0.05% formic acid, gradient: 0.0 min 100% A→2.9 min 30% A→3.1 min 10% A→4.5 min 10% A.

### Example A

Cellular Transactivation Assay:

Test Principle:

A cellular assay is used to identify activators of the peroxisome proliferator-activated receptor delta (PPAR-delta).

Since mammalian cells contain different endogenous nuclear receptors which may complicate an unambiguous interpretation of the results, an established chimera system is used in which the ligand binding domain of the human PPARδ receptor is fused to the DNA binding domain of the yeast transcription factor GAL4. The resulting GAL4-PPARδ chimera is co-transfected and stably expressed in CHO cells having a reporter construct.

Cloning:

The GAL4-PPARδ expression construct contains the ligand binding domain of PPARδ (amino acids 414-1326), which is PCR-amplified and cloned into the vector pcDNA3.1. This vector already contains the GAL4 DNA binding domain (amino acids 1-147) of the vector pFC2-dbd (Stratagene). The reporter construct, which contains five copies of the GAL4 binding site upstream of a thymidine kinase promoter, expresses firefly luciferase (Photinus pyralis) following activation and binding of GAL4-PPARδ.

Transactivation Assay (Luciferase Reporter):

CHO (chinese hamster ovary) cells are sown in CHO-A-SFM medium (GIBCO), supplemented by 2.5% foetal calf serum, 1% penicillin/streptomycin (GIBCO), at a cell density of 2×10³ cells per well in a 384 well plate (Greiner). The cells are cultivated at 37° C. for 48 h and then stimulated. To this end, the substances to be tested are taken up in the abovementioned medium and added to the cells. After a stimulation period of 24 hours, the luciferase activity is measured using a video camera. The relative light units measured give, as a function of the substance concentration, a sigmoidal stimulation curve. The EC₅₀ values are calculated using the computer program GraphPad PRISM (Version 3.02).

In this test, the compounds according to the invention of Examples 1, 2, 3, 4, 6, 8 and 9 show EC₅₀ values of from 1 to 100 nM.

### Example B

Description of the Test for Finding Pharmacologically Active Substances which Increase HDL Cholesterol (HDL-C) Concentrations in the Serum of Transgenic Mice Transfected with the Human ApoA1 Gene (hApoA1) and/or Have an Effect on the Metabolic Syndrome of Adipose ob,ob Mice and Lower their Blood Glucose Concentration:

The substances to be examined in vivo for their HDL-C-increasing activity are administered orally to male transgenic hApoA1 mice. One day prior to the start of the experiment, the animals are randomized into groups with the same number of animals, generally n=7-10. During the entire experiment, the animals have drinking water and feed ad libitum. The substances are administered orally once a day for 7 days; To this end, the test substances are dissolved in a solution of Solutol HS 15+ethanol+saline (0.9%) in a ratio of 1+1+8 or in a solution of Solutol HS 15+saline (0.9%) in a ratio of 2+8. The dissolved substances are administered in a volume of 10 ml/kg of body weight using a stomach tube. Animals which have been treated in exactly the same manner but have only been given the solvent (10 ml/kg of body weight), without test substance, serve as control group.

Prior to the first administration of substance, a blood sample from each of the mice is taken by puncture of the retroorbital venous plexus, to determine ApoA1, serum cholesterol, HDL-C and serum triglycerides (TG) (zero value). Subsequently, using a stomach tube, the test substance is administered for the first time to the animals. 24 hours after the last administration of substance (on day 8 after the start of the treatment), another blood sample is taken from each animal by puncture of the retroorbital venous plexus, to determine the same parameters. The blood samples are centrifuged and, after the serum has been obtained, cholesterol and TG are determined photometrically using an EPOS Analyzer 5060 (Eppendorf-Geratebau, Netheler & Hinz GmbH, Hamburg). The said determinations are carried out using commercial enzyme tests (Boehringer Mannheim, Mannheim).

To determine the HDL-C, the non-HDL-C fraction is precipitated using 20% PEG 8000 in 0.2 M glycine buffer pH 10. From the supernatant, the cholesterol is determined UV-photometrically (BIO-TEK Instruments, USA) in a 96-well plate using a commercial reagent (Ecoline 25, Merck, Darmstadt).

Human mouse ApoA1 is determined with a Sandwich ELISA method using a polyclonal antihuman ApoA1 and a monoclonal antihuman ApoA1 antibody (Biodesign International, USA). Quantification is carried out UV-photometrically (BIO-TEK Instruments, USA) using peroxidase-coupled anti-mouse-IGG antibodies (KPL, USA) and peroxidase substrate (KPL, USA).

The effect of the test substances on the HDL-C concentration is determined by subtracting the value measured for the 1^{st} blood sample (zero value) from the value measured for the 2^{nd} blood sample (after the treatment). The mean of the differences of all HDL-C values of one group is determined and compared to the mean of the differences of the control group.

Statistical evaluation is carried out using Student's t-test, after the variances have been checked for homogeneity.

Substances which increase the HDL-C of the treated animals in a statistically significant (p<0.05) manner by at least 15%, compared to the control group, are considered to be pharmacologically effective.

To examine substances for their effect on a metabolic syndrome, animals having an insulin resistance and increased blood glucose levels are used. To this end, C57Bl/6J Lep <ob> mice are treated using the same protocol as for the transgenic ApoA1 mice. The serum lipids are determined as described above. In these animals, serum glucose is additionally determined, as a parameter for blood glucose. Serum glucose is determined enzymatically in an EPOS Analyzer 5060 (see above), using commercially available enzyme tests (Boehringer Mannheim).

A blood-glucose-lowering effect of the test substances is determined by subtracting the value measured for the 1st blood sample (zero value) from the value measured for the 2nd blood sample of the same animal (after the treatment). The mean of the differences of all serum glucose values of one group is determined and compared to the mean of the differences-of the control group.

Statistical evaluation is carried out using Student's t-test, after the variances have been checked for homogeneity.

Substances which lower the serum glucose concentration of the treated animals in a statistically significant (p<0.05) manner by at least 10%, compared to the control group, are considered to be pharmacologically effective.

1. A compound of formula (I) 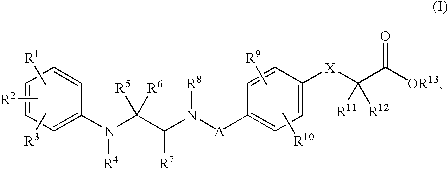
in which
A represents a bond or represents a —CH₂— or —CH₂CH₂— group,
X represents O, S or CH₂,
R¹, R² and R³ are identical or different and independently of one another represent hydrogen, (C₁-C₆)-alkyl, (C₃-C₇)-cycloalkyl, hydroxyl, (C₁-C₆)-alkoxy, amino, mono- or di-(C₁-C₆)-alkylamino, halogen, trifluoromethyl, trifluoromethoxy, nitro or cyano,
R⁴ represents hydrogen or (C₁-C₄)-alkyl,
R⁵ and R⁶ are hydrogen or together with the carbon atom to which they are attached form a carbonyl group,
R⁷ represents hydrogen or (C₁-C₄)-alkyl,
R⁸ represents straight-chain (C₅-C₁₀)-alkyl or represents a group of the formula —(CH₂)ₙ-E, in which
E represents (C₃-C₁₂)-cycloalkyl which may be substituted up to four times by identical or different radicals from the group consisting of (C₁-C₆)-alkyl, trifluoromethyl, hydroxyl, (C₁-C₆)-alkoxy, carboxyl and (C₁-C₆)-alkoxycarbonyl, or represents 4- to 8-membered heterocyclyl which has up to two heteroatoms from the group consisting of O and S and which may be substituted up to two times by identical or different radicals from the group consisting of (C₁-C₆)-alkyl, and
n represents the number 0, 1 or 2,
R⁹ and R¹⁰ are identical or different and independently of one another represent hydrogen, (C₁-C₆)-alkyl, (C₁-C₆)-alkoxy, trifluoromethyl or halogen,
R¹¹ and R¹² are identical or different and independently of one another represent hydrogen or (C₁-C₄)-alkyl, and
R¹³ represents hydrogen or a hydrolysable group which can be degraded to the corresponding carboxylic acid,
or a pharmaceutically acceptable salt hydrate or solvate thereof.

2. A compound of formula (I) according to claim 1,
in which
A represents a —CH₂— or —CH₂CH₂— group,
X represents O or S,
R¹ and R² are identical or different and independently of one another represent hydrogen, (C₁-C₄)-alkyl, di-(C₁-C₄)-alkylamino, chlorine, fluorine, trifluoromethyl, trifluoromethoxy, nitro or cyano,
R³ represents hydrogen,
R⁴ represents hydrogen or methyl,
R⁵ and R⁶ represent hydrogen or together with the carbon atom to which they are attached form a carbonyl group,
R⁷ represents hydrogen,
R⁸ represents (C₃-C₈)-cycloalkyl, which may be substituted up to four times by identical or different substituents from the group consisting of (C₁-C₄)-alkyl, trifluoromethyl, (C₁-C₄)-alkoxy, carboxyl and (C₁-C₄)-alkoxycarbonyl or represents 5- or 6-membered heterocyclyl which has up to two heteroatoms from the group consisting of O and S and which may be substituted up to two times by identical or different substituents from the group consisting of (C₁-C₄)-alkyl,
R⁹ represents hydrogen, (C₁-C₃)-alkyl, (C₁-C₃)-alkoxy, trifluoromethyl, fluorine or chlorine,
R¹⁰ represents hydrogen,
R¹¹ and R¹² are identical or different and independently of one another represent hydrogen or methyl, and
R¹³ represents hydrogen or represents a hydrolysable group which can be degraded to the corresponding carboxylic acid,
or a pharmaceutically acceptable salt hydrate or solvate thereof.

3. A compound of formula (I) according to claim 1
in which
A represents a —CH₂— group,
X represents O or S,
R¹ represents hydrogen, methyl, trifluoromethyl, chlorine, fluorine, nitro or cyano,
R² represents methyl, trifluoromethyl, chlorine, fluorine, nitro or cyano,
R³ represents hydrogen,
R⁴ represents hydrogen,
R⁵ and R⁶ together with the carbon atom to which they are attached form a carbonyl group,
R⁷ represents hydrogen,
R⁸ represents cyclopentyl or cyclohexyl, each of which may be substituted by methoxy, ethoxy or up to four times by methyl, or represents 3-tetrahydrofuranyl, 3-tetrahydropyranyl or 4-tetrahydropyranyl, each of which may be mono- or disubstituted by methyl,
R⁹ represents methyl,
R¹⁰ represents hydrogen,
R¹¹ and R¹² both represent hydrogen or represent methyl, and
R¹³ represents a hydrolysable group which can be degraded to the corresponding carboxylic acid, or represents hydrogen,
or a pharmaceutically acceptable salt hydrate or solvate thereof.

4. A compound of formula (I) according to claim 1 in which R⁴ represents hydrogen or methyl and R⁷ represents hydrogen.

5. A compound of formula (I) according to claim 1 in which R⁵ and R⁶ together with the carbon atom to which they are attached form a carbonyl group.

6. A compound of formula (IA) 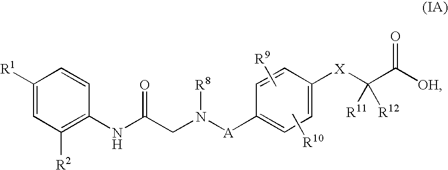
in which
R¹ and R² are identical or different and independently of one another represent methyl, trifluoromethyl, fluorine, chlorine, nitro or cyano, and
A, X, R⁸, R⁹, R¹⁰, R¹¹ and R¹² each have the meanings given in claim 1.

7. (canceled)

8. A pharmaceutical composition, comprising at least one compound of the formula (I) or (IA) as defined in claim 1 or 6, and inert non-toxic pharmaceutically acceptable carriers, auxiliaries, solvents, vehicles, emulsifiers and/or dispersants.

9. (canceled)

10. (canceled)

11. A method of treating coronary heart diseases, dyslipidaemias or restenosis after coronary angioplasty or stenting, comprising administering to a patient in need thereof an effective amount of a compound of the formula (I) as defined in claim 1 or an effective amount of a compound of the formula (IA) as defined in claim 6.

12. (canceled)

13. A process for preparing a pharmaceutical composition, comprising converting at least one compound of the formula (I) or (IA) as defined in claim 1 or 6 into an administration form using auxiliaries and/or carriers.

14. Process for preparing compounds of the formula (I) as defined in claim 1, characterized in that
[A] compounds of formula (II) 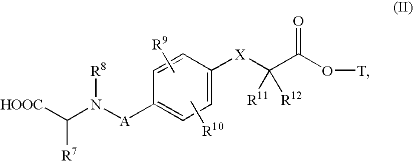
in which
A, X, R⁷, R⁸, R⁹, R¹⁰, R¹¹ and R¹² are as defined in claim 1 and
T represents benzyl, (C₁-C₆)-alkyl or a polymeric support suitable for solid-phase synthesis,
are initially reacted, with activation of the carboxylic acid group in (II), with compounds of formula (III) 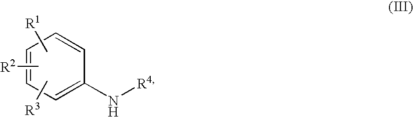
in which
R¹, R², R³ and R⁴ are as defined in claim 1
to give compounds of formula (Ia) 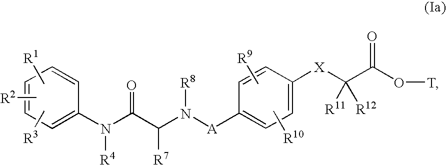
in which
A, X, T, R¹, R², R³, R⁴, R⁷, R⁸, R⁹, R¹⁰, R¹¹ and R¹² are as defined above, or
[B] compounds of formula (IV) 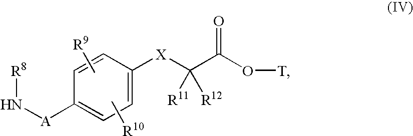
in which
A, X, T, R⁸, R⁹, R¹⁰, R¹¹ and R¹² are as defined in claim 1
are reacted in the presence of a base with compounds of formula (V) 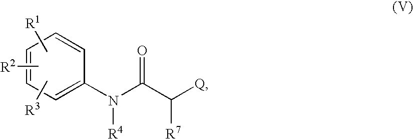
in which
R¹, R², R³, R⁴ and R⁷ are as defined in claim 1 and
Q represents a suitable leaving group,
likewise to give compounds of formula (Ia),
the compounds of formula (Ia) are then optionally converted by known methods for the reduction of amides into compounds of the formula (Ib) 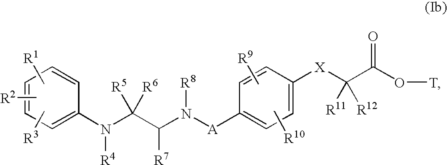
in which
A, X, T, R¹, R², R³, R⁴, R⁵, R⁶, R⁷, R⁸, R⁹, R¹⁰, R¹¹ and R¹² are as defined above,
which are subsequently converted with acids or bases into the corresponding carboxylic acids of formula (Ic) 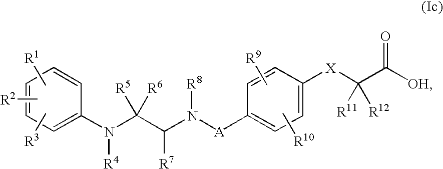
in which
A, X, R¹, R², R³, R⁴, R⁵, R⁶, R⁷, R⁸, R⁹, R¹⁰, R¹¹ and R¹² are as defined above,
and these are optionally further modified by known esterification methods by reaction with compounds of formula (VI)
R¹³-Z (VI),
in which
R¹³ is as defined in claim 1 and
Z represents a suitable leaving group, or represents a hydroxyl group.

15. A method of preventing coronary heart diseases, dyslipidaemias, or myocardial infarction, comprising administering to a patient in need thereof an effective amount of a compound of formula (I) as defined in claim 1 or an effective amount of a compound of the formula (IA) as defined in claim 6.
